(19) United States
(12) Patent Application Publication
(10) Pub. No.: US 2019/0256929 A1
(43) Pub. Date: Aug. 22, 2019

(54) PHARMACODYNAMIC BIOMARKERS FOR PERSONALIZED CANCER CARE USING EPIGENETIC MODIFYING AGENTS

(71) Applicant: ORYZON GENOMICS, S.A., Madrid (ES)

(72) Inventors: Fabian BIRZELE, Basel (CH); Wei-Yi CHENG, New York, NY (US); Mark D. DEMARIO, New York, NY (US); Fiona MACK, New York, NY (US); Francesca MILLETTI, New York, NY (US); William E. PIERCEALL, New York, NY (US)

(21) Appl. No.: 16/346,909

(22) PCT Filed: Nov. 2, 2017
(86) PCT No.: PCT/EP2017/077994

§ 371 Date: May 2, 2019

(30) Foreign Application Priority Data
Nov. 3, 2016 (EP) 16197012.4

(51) Int. Cl. C12Q 1/6886; G16H 50/20; G01N 33/50

(52) U.S. Cl. CPC C12Q 1/6886; G16H 50/20; C12Q 2600/156; C12Q 2600/106; C12Q 2600/158; G01N 33/5008

(57) ABSTRACT

The invention provides methods of monitoring differential gene expression of pharmacodynamic (PD) biomarkers in patients treated with Lysine Demethylase 1 (LSD 1) inhibitors and methods of determining the sensitivity of a cell to an LSD 1 inhibitor by measuring PD biomarkers.

### FIELD OF THE INVENTION

The invention provides methods of monitoring differential gene expression of pharmacodynamic (PD) biomarkers in patients treated with Lysine Demethylase 1 (LSD1) inhibitors and methods of determining the sensitivity of a cell to an LSD1 inhibitor by measuring PD biomarkers.

### BACKGROUND OF THE INVENTION

Aberrant gene expression in affected tissue as compared to normal tissue is a common characteristic of many human diseases. This is true for cancer and many neurological diseases which are characterized by changes in gene expression patterns. Gene expression patterns are controlled at multiple levels in the cell. Control of gene expression can occur through modifications of DNA: DNA promoter methylation is associated with suppression of gene expression. Several inhibitors of DNA methylation are approved for clinical use including the blockbuster Vidaza™. Another class of modifications involve histones which form the protein scaffold that DNA is normally associated with (coiled around) in eukaryotic cells. Histones play a crucial role in organizing DNA and the regulated coiling and uncoiling of DNA around the histones is critical in controlling gene expression—coiled DNA is typically not accessible for gene transcription. A number of histone modifications have been discovered including histone acetylation, histone lysine methylation, histone arginine methylation, histone ubiquinylation, and histone sumoylation, many of which modify accessibility to the associated DNA by the cells transcriptional machinery. These histone marks serve to recruit various protein complexes involved in transcription and repression. An increasing number of studies are painting an intricate picture of how various combinations of histone marks control gene expression in cell-type specific manner and a new term has been coined to capture this concept: the histone code.

The prototypical histone mark is histone acetylation. Histone acetyl transferase and histone deacetylases are the catalytic machines involved in modulation of this histone mark although typically these enzymes are parts of multiprotein complexes containing other proteins involved in reading and modifying histone marks. The components of these protein complexes are typically cell-type specific and typically comprise transcriptional regulators, repressors, co-repressors, receptors associated with gene expression modulation (e.g., estrogen or androgen receptor). Histone deacetylase inhibitors alter the histone acetylation profile of chromatin. Accordingly, histone deacetylase inhibitors like Vorinostat (SAHA), Trichostatin A (TSA), and many others have been shown to alter gene expression in various in vitro and in vivo animal models. Clinically, histone deacetylase inhibitors have demonstrated activity in the cancer setting and are being investigated for oncology indications as well as for neurological conditions and other diseases.

Another modification that is involved in regulating gene expression is histone methylation including lysine and arginine methylation. The methylation status of histone lysines has recently been shown to be important in dynamically regulating gene expression.

A group of enzymes known as histone lysine methyl transferases and histone lysine demethylases are involved in histone lysine modifications. One particular human histone lysine demethylase enzyme called Lysine Specific Demethylase-1 (LSD1) was recently discovered (Shi et al. (2004) Cell 119:941) to be involved in this crucial histone modification. LSD1 has a fair degree of structural similarity, and amino acid identity/homology to polyamine oxidases and monoamine oxidases, all of which (i.e., MAO-A, MAO-B and LSD1) are flavin dependent amine oxidases which catalyze the oxidation of nitrogen-hydrogen bonds and/or nitrogen carbon bonds. LSD1 has been recognized as an interesting target for the development of new drugs to treat cancer, neurological diseases and other conditions.

LSD1 is a flavin-containing amino oxidase (AO) that specifically catalyzes the demethylation of mono- and di-methylated histone H3 lysine 4 (H3K4me1/me2). LSD1 is described as a key histone modifier involved in the maintenance of pluripotency in stem cells by regulating the critical balance between H3K4 and H3K27 methylation at their regulatory regions (Adamo A. et al. (2011) Nature Cell Biology 13:652-659). In the context of oncogenic gene programs, LSD1 has been reported to possess oncogenic properties in several cancer types, while its inhibition reduces or blocks cell growth (Amente S. et al. (2013) Biochimica et Biophysica Acta 1829(10):981-986). Multiple preclinical studies have provided preclinical proof of concept for using LSD1 inhibitor to treat acute leukemia (Harris W. J. et al. (2012) Cancer Cell 21:473-487, Schenk T. et al. (2012) Nat. Med. 18:605-611) and small cell lung cancer (Mohammad H. P. et al. (2015) Cancer Cell 28(1):57-69).

Cyclopropylamine containing compounds are known to inhibit a number of medically important targets including amine oxidases like Monoamine Oxidase A (MAO-A; or MAOA), Monoamine Oxidase B (MAO-B; or MAOB), and Lysine Specific Demethylase-1 (LSD1). Tranylcypromine (also known as 2-phenylcyclopropylamine), which is the active ingredient of Parnate® and one of the best known examples of a cyclopropylamine, is known to inhibit all of these enzymes. Since MAO-A inhibition may cause undesired side effects, it would be desirable to identify cyclopropylamine derivatives that exhibit potent LSD1 inhibitory activity while being devoid of or having substantially reduced MAO-A inhibitory activity.

Compounds which act as inhibitors of LSD1 are known in the art. LSD1 inhibitors and methods for making them are for example disclosed in WO 2011/131697 (A1), WO 2012135113 (A2), WO 2013/057322 (A1), WO 2010/143582, WO 2011/131576, WO 2013/022047, WO 2013/025805, WO 2014/058071, WO 2014/084298, WO 2014/085613, WO 2014/086790, WO2014/164867, WO 2014/194280, WO 2014/205213, WO 2015/021128, WO 2015/031564, WO 2015/089192, WO 2015/120281, WO 2015/123465, WO 2015/123437, WO 2015/123424, WO 2015/123408, WO 2015/134973, WO 2015/156417 and WO 2015/168466 which are incorporated in their entirety herein.

WO 2012135113 (A2) discloses compounds, for example GSK2879552 [CAS Reg. No. 1401966-69-5], also known as 4-[[4-[[[(1R,2S)-2-phenylcyclopropyl]amino]methyl]-1-piperidinyl]methyl]-benzoic acid (Example 26 on p. 75, Example 29 on p. 81), as selective LSD1 inhibitor.

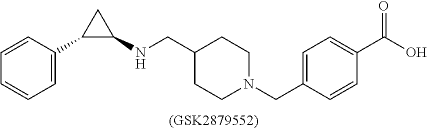 [SMILES: O=C(O)c1ccc(CN2CCC(CN[C@@H]3C[C@H]3c3ccccc3)CC2)cc1]

LSD1 inhibitors and methods for making them are for example disclosed in WO 2011/131697 (A1), particularly examples 1-21 (pages 90 to 103), which are incorporated in their entirety herein.

LSD1 inhibitors and methods for making them are for example disclosed in WO 2013/057322 (A1), particularly examples 1-108 (pages 155 to 191), which are incorporated in their entirety herein.

Particular LSD1 inhibitors described in WO 2013/057322 (A1) are provided in Table 1.

TABLE 1
Particular LSD1 inhibitors disclosed in WO 2013/057322 (A1).

| Example No of | | |
|---|---|---|
| WO 2013/057322 | Substance name | Structure |
| 1 | N1-((trans)-2-phenylcyclopropyl) cyclohexane-1,4-diamine | 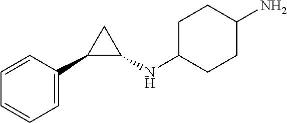 [SMILES: NC1CCC(N[C@H]2C[C@@H]2c2ccccc2)CC1] |
| 5 | (trans)-N1-((1R,2S)-2- phenylcyclopropyl) cyclohexane-1,4-diamine | 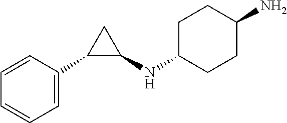 [SMILES: N[C@H]1CC[C@H](N[C@@H]2C[C@H]2c2ccccc2)CC1] |
| 15 | (R)-1-(4-(((trans)-2- phenylcyclopropyl)amino) cyclohexyl)pyrrolidin-3-amine | 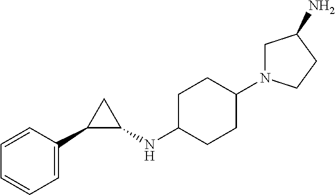 [SMILES: N[C@H]1CCN(C2CCC(N[C@H]3C[C@@H]3c3ccccc3)CC2)C1] |
| 17 | 4-(aminomethyl)-N-((trans)-2- phenylcyclopropyl) cyclohexanamine | 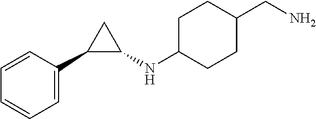 [SMILES: NCC1CCC(N[C@H]2C[C@@H]2c2ccccc2)CC1] |
| 18 | N1-((trans)-2-phenylcyclopropyl) cyclohexane-1,3-diamine | 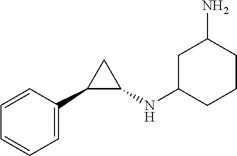 [SMILES: NC1CCCC(N[C@H]2C[C@@H]2c2ccccc2)C1] |
| 19 | N1-((trans)-2-phenylcyclopropyl) cyclobutane-1,3-diamine | 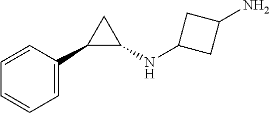 [SMILES: NC1CC(N[C@H]2C[C@@H]2c2ccccc2)C1] |
| 20 | N1-((trans)-2-phenylcyclopropyl)- 2,3-dihydro-1H-indene-1,3-diamine | 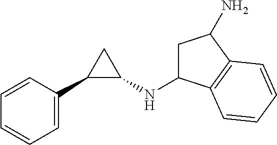 [SMILES: NC1CC(N[C@H]2C[C@@H]2c2ccccc2)c2ccccc21] |
| 22 | N1-methyl-N4-((trans)-2- phenylcyclopropyl) cyclohexane-1,4-diamine | 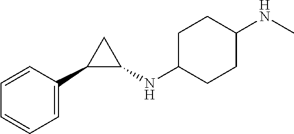 [SMILES: CNC1CCC(N[C@H]2C[C@@H]2c2ccccc2)CC1] |
| 26 | N1-((trans)-2-(4- bromophenyl)cyclopropyl) cyclohexane-1,4-diamine | 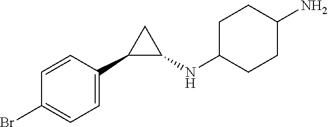 [SMILES: NC1CCC(N[C@H]2C[C@@H]2c2ccc(Br)cc2)CC1] |
| 27 | N1-(2-(o-tolyl)cyclopropyl) cyclohexane-1,4-diamine | 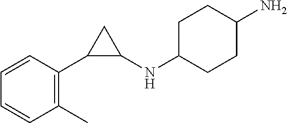 [SMILES: Cc1ccccc1C1CC1NC1CCC(N)CC1] |
| 29 | N1-(2-(4- methoxyphenyl)cyclopropyl) cyclohexane-1,4-diamine | 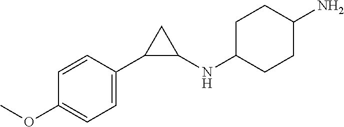 [SMILES: COc1ccc(C2CC2NC2CCC(N)CC2)cc1] |
| 31 | N1-(2-(2-fluorophenyl)cyclopropyl) cyclohexane-1,4-diamine | 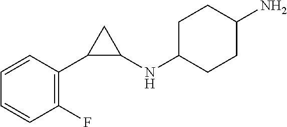 [SMILES: NC1CCC(NC2CC2c2ccccc2F)CC1] |
| 33 | N1-(2-(naphthalen-2- yl)cyclopropyl) cyclohexane-1,4-diamine | 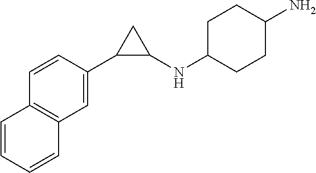 [SMILES: NC1CCC(NC2CC2c2ccc3ccccc3c2)CC1] |
| 50 | N-(4′-((trans)-2-((4- aminocyclohexyl)amino) cyclopropyl)-[1,1′-biphenyl]-3-yl)- 2-cyanobenzenesulfonamide | 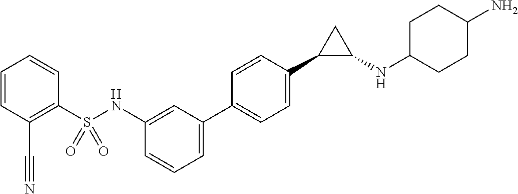 [SMILES: N#Cc1ccccc1S(=O)(=O)Nc1cccc(-c2ccc([C@H]3C[C@@H]3NC3CCC(N)CC3)cc2)c1] |
| 56 | N1-((trans)-2-(4-(pyridin-3- ylmethoxy)phenyl)cyclopropyl) cyclohexane-1,4-diamine | 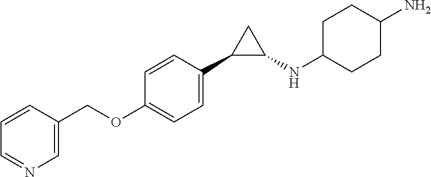 [SMILES: NC1CCC(N[C@H]2C[C@@H]2c2ccc(OCc3cccnc3)cc2)CC1] |

A more particular LSD1 inhibitor described in WO 2013/057322 (A1) is (trans)-N1-((1R,2S)-2-phenylcyclopropyl)cyclohexane-1,4-diamine [CAS Reg. No. 1431304-21-0]

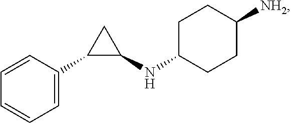 [SMILES: N[C@H]1CC[C@H](N[C@@H]2C[C@H]2c2ccccc2)CC1]

corresponding to Example 5 therein, and pharmaceutically acceptable salts thereof.

Even though potent selective LSD1 inhibitors have been proposed for adequate treatments for conditions such as cancer and neurodegeneration, biomarkers for personalized treatment have not been described.

It has long been acknowledged that there is a need to develop methods of individualizing cancer treatment. Pharmacodynamic (PD) markers that indicate whether a therapeutic is active can be useful to monitor the response of patients receiving such therapeutic. If a PD marker suggests that a patient is not responding appropriately to the treatment, then the dosage administered can be increased, decreased or completely discontinued. PD markers are thus useful in determining that patients receive the correct course of treatment.

In the development of LSD1 inhibitors, PD markers may also facilitate understanding of the drug's mechanism of action.

Further, degree of mechanism of action related PD changes may be correlated with drug exposure to determine effective dose and related PD changes as both are correlated with intended changes in oncology cellular growth dynamics changes.

Therefore, it is an aim of the present invention to provide pharmacodynamic biomarkers to monitor sensitivity of a cell to respond to LSD1 inhibitor treatment in patients with neoplastic diseases.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

All publications, patent applications, patents, and other references mentioned herein are incorporated by reference in their entirety.

The nomenclature used in this Application is based on IUPAC systematic nomenclature, unless indicated otherwise.

Any open valency appearing on a carbon, oxygen, sulfur or nitrogen atom in the structures herein indicates the presence of a hydrogen, unless indicated otherwise.

When indicating the number of substituents, the term “one or more” refers to the range from one substituent to the highest possible number of substitution, i.e. replacement of one hydrogen up to replacement of all hydrogens by substituents.

The term “optional” or “optionally” denotes that a subsequently described event or circumstance can but need not occur, and that the description includes instances where the event or circumstance occurs and instances in which it does not.

“The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511). The prefixes D and L or (+) and (−) are employed to designate the sign of rotation of plane-polarized light by the compound, with (−) or L designating that the compound is levorotatory. A compound prefixed with (+) or D is dextrorotatory.

The terms “pharmaceutical composition” and “pharmaceutical formulation” (or “formulation”) are used interchangeably and denote a mixture or solution comprising a therapeutically effective amount of an active pharmaceutical ingredient together with pharmaceutically acceptable excipients to be administered to a mammal, e.g., a human in need thereof.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “inhibitor” denotes a compound which competes with, reduces or prevents the binding of a particular ligand to a particular receptor or enzyme and/or which reduces or prevents the activity of a particular protein, e.g. of a receptor or an enzyme.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “animal” as used herein comprises human beings and non-human animals. In one embodiment, a “non-human animal” is a mammal, for example a rodent such as rat or a mouse. In one embodiment, a non-human animal is a mouse.

The term “half maximal effective concentration” (EC50) denotes the plasma concentration of a particular compound or molecule required for obtaining 50% of the maximum of a particular effect in vivo.

The term “therapeutically effective amount” (or “effective amount”) denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “treating” or “treatment” of a disease state includes inhibiting the disease state, i.e., arresting the development of the disease state or its clinical symptoms, or relieving the disease state, i.e., causing temporary or permanent regression of the disease state or its clinical symptoms.

The term “assessing a neoplastic disease” is used to indicate that the method according to the present invention will aid a medical professional including, e.g., a physician in assessing
whether an individual has a neoplastic disease or is at risk of developing a neoplastic disease;
the response of a patient having a neoplastic disease to therapy
efficacy of therapy in a patient having a neoplastic disease,
prognosing the course of a neoplastic disease.

In one embodiment the term assessing a neoplastic disease is used to indicate efficacy of therapy in a patient having a neoplastic disease.

The term “assessing a therapy” is used to indicate that the method according to the present invention will aid a medical professional including, e.g., a physician in assessing whether an individual having a neoplastic disease should be treated with an effective amount of an LSD1 inhibitor and how an effective amount of an LSD1 inhibitor can be adapted or optimized.

In certain embodiments, the term “up-regulated level” refers to an increase of an mRNA transcript expression level of a gene panel or an expression level of the translated protein of a gene panel measured in a sample from the patient after begin of the therapy as compared to the level measured prior to begin of the therapy, particularly to an increase of 5%, 10%, 20%, 25%, 30%, 40%, 50%, 60%, 70%, 80%, 85%, 90%, 95%, 100% or greater, determined by the methods described herein. In certain embodiments, the term “up-regulated level” refers to an increase in a level of the gene panel in the sample from the patient wherein the increase is at least about 1.5-, 1.75-, 2-, 3-, 4-, 5-, 6-, 7-, 8-, 9-, 10-, 15-, 20-, 25-, 30-, 40-, 50-, 60-, 70-, 75-, 80-, 90-, or 100-fold higher after begin of the therapy as compared to the level prior to begin of the therapy.

In certain embodiments, the term “down-regulated level” refers to a decrease of an mRNA transcript expression level of a gene panel or an expression level of the translated protein of a gene panel measured in a sample from the patient after begin of the therapy as compared to the level measured prior to begin of the therapy, particularly to a decrease of 5%, 10%, 20%, 25%, 30%, 40%, 50%, 60%, 70%, 80%, 85%, 90%, 95%, 100% or greater, determined by the methods described herein. In certain embodiments, the term “down-regulated level” refers to a decrease in a level of the gene panel in the sample from the patient wherein the decreased level is at most about 0.9-, 0.8-, 0.7-, 0.6-, 0.5-, 0.4-, 0.3-, 0.2-, 0.1-, 0.05-, or 0.01-fold after begin of the therapy as compared to the level prior to begin of the therapy.

In certain embodiments, the term “after begin of therapy” refers to a period of 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 1 d, 1.5 d, 2 d, 2.5 d, 3 d, 3.5 d, 4 d, 4.5 d, 5 d, 5.5 d, 6 d, 6.5 d, 7 d, 8 d, 9 d, 10 d, 11 d, 12 d, 13 d, 14 d, 15 d, 16 d, 17 d, 18 d, 19 d, 20 d, 21 d, 22 d, 23 d, 24 d, 25 d, 26 d, 27 d, 28 d, 29 d or 30 d after start of the therapy.

The term “biomarker” as used herein refers generally to a gene, the expression or presence of which in or on a mammalian tissue or cell can be detected by standard methods (or methods disclosed herein) and which may be predictive, diagnostic and/or prognostic for a mammalian cell's or tissue's sensitivity to treatment regimes based on LSD1 inhibition by e.g. an LSD1 inhibitor such as (trans)-N1-((1R,2S)-2-phenylcyclopropyl)cyclohexane-1,4-diamine bis-hydrochloride. In certain embodiments, the level of such a biomarker is determined to be higher or lower than that observed for a reference sample.

The term “comparing” as used herein refers to comparing the level of the biomarker in the sample from the individual or patient with the reference level of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer assisted. Thus, the comparison may be carried out by a computing device (e.g., of a system disclosed herein). The value of the measured or detected level of the biomarker in the sample from the individual or patient and the reference level can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references which are stored in a database by a computer program. The computer program may further evaluate the result of the comparison, i.e. automatically provide the desired assessment in a suitable output format. For a computer assisted comparison, the value of the determined amount may be compared to values corresponding to suitable references which are stored in a database by a computer program. The computer program may further evaluate the result of the comparison, i.e. automatically provides the desired assessment in a suitable output format.

The term “detecting” a biomarker as used herein refers to methods of detecting the presence of quantity of the biomarker in the sample employing appropriate methods of detection described elsewhere herein.

The term “measuring” the level of a biomarker, as used herein refers to the quantification of the biomarker, e.g. to determining the level of the biomarker in the sample, employing appropriate methods of detection described elsewhere herein.

The term “monitoring the efficacy of a therapy” is used to indicate that a sample is obtained at least once, including serially, from a patient before and/or under therapy with an LSD1 inhibitor and that gene panel levels are measured therein to obtain an indication whether the therapy is efficient or not.

In the monitoring of the efficacy of a therapy the gene panel levels are measured and in one embodiment compared to a reference value for the gene panel, or, in a further embodiment, it is compared to the gene panel levels in a sample obtained from the same patient at an earlier point in time, e.g. while said patient was already under therapy or before start of a therapy in said patient.

A “patient” or “subject” herein is any single human subject eligible for treatment who is experiencing or has experienced one or more signs, symptoms, or other indicators of a neoplastic disease. Intended to be included as a subject are any subjects involved in clinical research trials not showing any clinical sign of disease, or subjects involved in epidemiological studies, or subjects once used as controls. The subject may have been previously treated with an LSD1 inhibitor or another drug, or not so treated. The subject may be naïve to an additional drug(s) being used when the treatment herein is started, i.e., the subject may not have been previously treated with, for example, a therapy other than an LSD1 inhibitor at “baseline” (i.e., at a set point in time before the administration of a first dose of Drug D in the treatment method herein, such as the day of screening the subject before treatment is commenced). Such “naïve” subjects are generally considered to be candidates for treatment with such additional drug(s).

The phrase “providing a diagnosis/assessment” as used herein refers to using the information or data generated relating to the gene panel levels in a sample of a patient to diagnose/assess a neoplastic disease in the patient. The information or data may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level.

The phrase “recommending a treatment” as used herein refers to using the information or data generated relating to the gene panel levels in a sample of a patient to identify the patient as suitably treated or not suitably treated with a therapy. In some embodiment the therapy may comprise an LSD1 inhibitor. In some embodiments the phrase “recommending a treatment/therapy” includes the identification of a patient who requires adaptation of an effective amount of an LSD1 inhibitor being administered. In some embodiments recommending a treatment includes recommending that the amount of an LSD1 inhibitor being administered is adapted. The phrase “recommending a treatment” as used herein also may refer to using the information or data generated for proposing or selecting a therapy comprising an LSD1 inhibitor for a patient identified or selected as more or less likely to respond to the therapy comprising a LSD1 inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level. In some embodiments, the information or data includes an indication that the patient is suitably treated or not suitably treated with a therapy comprising an LSD1 inhibitor.

In certain embodiments, the term “reference level” herein refers to a predetermined value. In this context “level” encompasses the absolute amount, the relative amount or concentration as well as any value or parameter which correlates thereto or can be derived therefrom. As the skilled artisan will appreciate the reference level is predetermined and set to meet routine requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90%, 95% or 98%, respectively. These requirements may also be defined in terms of positive or negative predictive values. Nonetheless, based on the teaching given in the present invention it will always be possible for a skilled artisan to arrive at the reference level meeting those requirements. In one embodiment the reference level is determined in reference samples from healthy individuals. The reference level in one embodiment has been predetermined in reference samples from the disease entity to which the patient belongs. In certain embodiments the reference level can e.g. be set to any percentage between 25% and 75% of the overall distribution of the values in a disease entity investigated. In other embodiments the reference level can e.g. be set to the median, tertiles or quartiles as determined from the overall distribution of the values in reference samples from a disease entity investigated. In one embodiment the reference level is set to the median value as determined from the overall distribution of the values in a disease entity investigated. The reference level may vary depending on various physiological parameters such as age, gender or subpopulation, as well as on the means used for the determination of the gene panel levels referred to herein. In one embodiment, the reference sample is from essentially the same type of cells, tissue, organ or body fluid source as the sample from the individual or patient subjected to the method of the invention, e.g. if according to the invention blood is used as a sample to determine the gene panel levels in the individual, the reference level is also determined in blood or a part thereof.

The phrase “responsive to” in the context of the present invention indicates that a patient suffering from, being suspected to suffer or being prone to suffer from, or diagnosed with a disorder as described herein, shows a response to therapy comprising an LSD1 inhibitor.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, bronchial lavage or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation.

The phrase “selecting a patient” or “identifying a patient” as used herein refers to using the information or data generated relating to the gene panel levels in a sample of a patient to identify or selecting the patient as more likely to benefit or less likely to benefit from a therapy comprising an LSD1 inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level. In some embodiments, the information or data includes an indication that the patient is more likely or less likely to respond to a therapy comprising an LSD1 inhibitor.

The phrase “selecting a therapy” as used herein refers to using the information or data generated relating to the gene panel levels in a sample of a patient to identify or selecting a therapy for a patient. In some embodiment the therapy may comprise an LSD1 inhibitor. In some embodiments the phrase “identifying/selecting a therapy” includes the identification of a patient who requires adaptation of an effective amount of an LSD1 inhibitor being administered. In some embodiments recommending a treatment includes recommending that the amount of LSD1 inhibitor being administered is adapted. The phrase “recommending a treatment” as used herein also may refer to using the information or data generated for proposing or selecting a therapy comprising an LSD1 inhibitor for a patient identified or selected as more or less likely to respond to the therapy comprising an LSD1 inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level. In some embodiments, the information or data includes an indication that a therapy comprising an LSD1 inhibitor is suitable for the patient.

In this application, the term “readout levels” denotes a value which can be in any form of mRNA expression measurement, such as for example expression levels derived from RNA-sequencing such as normalized read counts and RPKM (Reads per Kilobase of Million mapped reads); RT-qPCR; or microarrays. Alternatively the readout levels denotes a value which can be in the form of expression levels of translated proteins.

In this application, the term “normalized read count” denotes the read count which is obtained directly from a RNA-sequencing experiment and which is normalized to make it comparable across experiments.

In this application, the term “normalized expression level” denotes a value which is obtained in a particular kind of expression measurement and which is normalized to make it comparable across experiments (e.g. normalized expression from microarrays, normalized expression from RNA-sequencing).

The baseline expression levels of the genes of the gene panel may yield, alone or in combination with one another, a composite score to evaluate the response of a patient to LSD1 inhibitor containing therapy regimens. Combining the expression levels of genes may provide a multi-gene signature with improved confidence regarding responsiveness as compared to the readout from single gene expression levels.

The present invention identifies a gene panel whose mRNA transcript expression levels and/or the expression levels of the translated proteins may serve to assess the response of a patient to a therapy comprising an LSD1 inhibitor.

The mRNA transcript expression level of one gene of the gene panel, the mRNA transcript expression levels of a combination of two or more genes of the gene panel, the expression level of one protein translated from a gene of the gene panel, and/or the expression levels of a combination of two or more proteins translated from genes of the gene panel may serve to evaluate the response of a patient to a therapy comprising an LSD1 inhibitor.

In particular the invention relates to the up-regulation or down-regulation of the expression of the identified genes after LSD1 treatment.

The present invention identifies mRNAs associated with and for identifying responses to LSD1 inhibition. For example, the PD biomarkers ASCL1 and GRP exhibit down-regulated expression and the PD biomarker NOTCH1, DENND5A, CNN2, ZFP36L1, and VIM exhibit up-regulated expression in LSD1 inhibitor responsive cell lines versus non-responsive cell lines.

One embodiment of the invention provides an in vitro method of assessing the response of a patient having a neoplastic disease to a therapy comprising an LSD1 inhibitor, the method comprising steps:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels as measured in a) of the gene panel,
c) comparing the levels of the gene panel measured in a) to the levels of the gene panel measured in b), and
d) identifying the patient as responding to the therapy when the levels of the gene panel measured in b) are up-regulated or down-regulated as compared to the levels of the gene panel measured in a).

One embodiment of the invention provides an in vitro method of assessing the response of a patient having a neoplastic disease to a therapy comprising an LSD1 inhibitor, the method comprising:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels as measured in a) of the gene panel,
c) comparing the levels of the gene panel measured in a) to the levels of the gene panel measured in b),
d) identifying the patient as responding to the therapy when the levels of the gene panel measured in b) are up-regulated or down-regulated as compared to the levels of the gene panel measured in a), and
e) optimizing the therapy by recommending that the patient be treated with an adapted effective amount of LSD1 inhibitor.

Another embodiment of the invention provides an in vitro method of monitoring efficacy of therapy comprising an LSD1 inhibitor in a patient having a neoplastic disease, the method comprising steps:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels as measured in a) of the gene panel,
c) comparing the levels of the gene panel measured in a) to the levels of the gene panel measured in b), and
d) identifying the patient as responding to the therapy when the levels of the gene panel measured in b) are up-regulated or down-regulated as compared to the levels of the gene panel measured in a).

Another embodiment of the invention provides an in vitro method of monitoring efficacy of therapy comprising an LSD1 inhibitor in a patient having a neoplastic disease, the method comprising steps:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels as measured in a) of the gene panel,
c) comparing the levels of the gene panel measured in a) to the levels of the gene panel measured in b), and
d) identifying the patient as responding to the therapy when the levels of the gene panel measured in b) are up-regulated or down-regulated as compared to the levels of the gene panel measured in a), and
e) optimizing the therapy by recommending that the patient be treated with an adapted effective amount of LSD1 inhibitor.

Another embodiment of the invention provides a method of treating a patient having a neoplastic disease, the method comprising:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels of the gene panel,
c) comparing the levels of the gene panel measured in a) to the levels of the gene panel measured in b), and
d) identifying the patient as responding to the therapy when the levels of the gene panel measured in b) are up-regulated or down-regulated as compared to the levels of the gene panel measured in a), and
e) administering an effective amount of LSD1 inhibitor to the patient if likely to respond thereby treating the neoplastic disease.

Another embodiment of the invention provides a method of treating a patient having a neoplastic disease, the method comprising:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels of the gene panel,
c) comparing the levels of the gene panel measured in a) to the levels of the gene panel measured in b), and
d) identifying the patient as responding to the therapy when the levels of the gene panel measured in b) are up-regulated or down-regulated as compared to the levels of the gene panel measured in a),
e) optimizing the therapy by recommending that the patient be treated with an adapted effective amount of LSD1 inhibitor, and
f) administering the adapted effective amount of LSD1 inhibitor to the patient if likely to respond thereby treating the neoplastic disease.

Another embodiment of the invention provides an LSD1 inhibitor for use in treating a patient having a neoplastic disease, wherein the patient is treated if one or more mRNA transcript expression levels of a gene panel and/or one or more expression levels of the translated proteins of a gene panel measured in a sample from the patient after begin of the therapy are up-regulated or down-regulated as compared to the levels measured prior to begin of the therapy thereby treating the neoplastic disease, wherein the gene panel comprises one or more genes.

Another embodiment of the invention provides an in vitro use of a gene panel comprising one or more genes for assessing a therapy comprising an LSD1 inhibitor in a patient having a neoplastic disease, wherein up-regulation or down-regulation of one or more mRNA transcript expression levels of a gene panel and/or one or more expression levels of the translated proteins of a gene panel measured in a sample from the patient after begin of the therapy as compared to the levels measured prior to begin of the therapy indicate that the patient should be treated with an effective amount of an LSD1 inhibitor.

Another embodiment of the invention provides an in vitro use of a gene panel comprising one or more genes for monitoring efficacy of therapy comprising an LSD1 inhibitor in a patient having a neoplastic disease, wherein up-regulation or down-regulation of one or more mRNA transcript expression levels of a gene panel and/or one or more expression levels of the translated proteins of a gene panel measured in a sample from the patient after begin of the therapy as compared to the levels measured prior to begin of the therapy indicate that the patient should be treated with an effective amount of an LSD1 inhibitor.

Another embodiment of the invention provides a use of a gene panel comprising one or more genes for the manufacture of a diagnostic for assessing a neoplastic disease.

Another embodiment of the invention provides a use of a gene panel comprising one or more genes for the manufacture of a diagnostic for assessing a therapy comprising an LSD1 inhibitor in a patient having a neoplastic disease.

Another embodiment of the invention provides a use of a gene panel comprising one or more genes for the manufacture of a diagnostic for monitoring efficacy of therapy comprising an LSD1 inhibitor in a patient having a neoplastic disease.

Another embodiment of the invention provides a kit for monitoring efficacy of therapy comprising an LSD1 inhibitor in a patient having a neoplastic disease comprising one or more reagents for measuring one or more mRNA transcript expression levels of a gene panel and/or one or more expression levels of the translated proteins of a gene panel in a sample, wherein the gene panel comprises one or more genes.

Another embodiment of the invention provides a method as described herein, an LSD1 inhibitor as described herein, in particular (trans)-N1-((1R,2S)-2-phenylcyclopropyl)cyclohexane-1,4-diamine, a use as described herein, or a kit as described herein, wherein the sample is taken from a whole blood specimen, a blood serum specimen, a blood plasma specimen, a bone marrow specimen, a saliva specimen, a skin specimen, a hair specimen, a fresh, frozen or formalin-fixed paraffin embedded primary human tumor specimen, a fresh, frozen or formalin-fixed paraffin embedded non-primary tumors, in particular metastases, ascites or circulating tumor cells.

Another embodiment of the invention provides a method as described herein, a LSD1 inhibitor as described herein, in particular (trans)-N1-((1R,2S)-2-phenylcyclopropyl)cyclohexane-1,4-diamine, a use as described herein, or a kit as described herein, wherein the gene panel comprises the NOTCH1 gene, wherein up-regulated levels of NOTCH1 after begin of therapy comprising the LSD1 inhibitor as described herein, in particular (trans)-N1-((1R,2S)-2-phenylcyclopropyl)cyclohexane-1,4-diamine, are indicative for a response of the patient to the therapy.

Table 2 provides a list including description of the genes of the gene panel as referred to in present invention.

TABLE 2
Description of the genes employed in the gene panel of the invention
(*http://www.ensembl.org/, Cunningham F. et al., Nucl. Acids Res. (2015) 43(D1): D662-D669).

| | | | | Location: |
|---|---|---|---|---|
| Gene | Ensembl Gene ID* | Description | Synonyms | Chromosome |
| ASCL1 | ENSG00000139352 | achaete-scute family | ASH1, bHLHa46, | Chromosome 12: |
| | | bHLH transcription | HASH1 | 102,957,686-102,960,516 |
| | | factor 1 | | forward strand. |
| CNN2 | ENSG00000064666 | calponin 2 | | Chromosome 19: |
| | | | | 1,026,581-1,039,068 |
| | | | | forward strand. |
| DENND5A | ENSG00000184014 | DENN domain | FLJ43455, | Chromosome 11: |
| | | containing 5A | FLJ33829, | 9,138,825-9,265,390 |
| | | | KIAA1091, | reverse strand. |
| | | | FLJ22354, RAB6IP1 | |
| GRP | ENSG00000134443 | gastrin-releasing | BN, proGRP, GRP- | Chromosome 18: |
| | | peptide | 10, preproGRP | 59,220,168-59,230,774 |
| | | | | forward strand. |
| NOTCH1 | ENSG00000148400 | notch 1 | AOVD1, AOS5, | Chromosome 9: |
| | | | hN1, TAN1 | 136,494,444-136,545,862 |
| | | | | reverse |
| | | | | strand. |
| VIM | ENSG00000026025 | vimentin | HEL113, CTRCT30 | Chromosome 10: |
| | | | | 17,228,259-17,237,593 |
| | | | | forward strand. |
| ZFP36L1 | ENSG00000185650 | ZFP36 ring finger | BERG36, TIS11B, | Chromosome 14: |
| | | protein like 1 | ERF1, Berg36, | 68,787,660-68,796,253 |
| | | | cMG1, BRF1, ERF- | reverse strand. |
| | | | 1, RNF162B | |

In one aspect of the invention, the levels measured are mRNA transcript expression levels.

In one aspect of the invention, the levels measured are mRNA transcript expression levels derived from RNA-sequencing, RT-qPCR or microarrays.

In one aspect of the invention, the levels measured are expression levels of translated proteins.

The gene panel comprises one or more genes selected from ASCL1, CNN2, DENND5A, GRP, NOTCH1, VIM, and ZFP36L1 (as described in Table 2).

The gene panel comprises one or more genes selected from NOTCH1, ASCL1, GRP, CNN2, DENND5A, VIM, and ZFP36L1 (as described in Table 2).

In a particular embodiment of the invention the gene panel comprises one or more genes selected from the group of ASCL1, CNN2, DENND5A, GRP, NOTCH1, VIM, and ZFP36L1.

In a particular embodiment of the invention the gene panel comprises one or more genes selected from the group of NOTCH1, ASCL1, GRP, CNN2, DENND5A, VIM, and ZFP36L1.

In a particular embodiment of the invention the gene panel comprises one or more genes selected from the group of CNN2, DENND5A, NOTCH1, VIM, and ZFP36L1.

In a particular embodiment of the invention the gene panel comprises one or more genes selected from the group of NOTCH1, CNN2, DENND5A, VIM, and ZFP36L1.

In a particular embodiment of the invention the gene panel comprises two, three, four or five genes selected from the group of ASCL1, CNN2, DENND5A, GRP, NOTCH1, and ZFP36L1.

In a particular embodiment of the invention the gene panel comprises two, three, four or five genes selected from the group of NOTCH1, ASCL1, GRP, CNN2, DENND5A, and ZFP36L1.

In a particular embodiment of the invention the gene panel comprises one or more genes selected from the group of CNN2, DENND5A, NOTCH1, and ZFP36L1.

In a particular embodiment of the invention the gene panel comprises one or more genes selected from the group of NOTCH1, CNN2, DENND5A, and ZFP36L1.

In a particular embodiment of the invention the gene panel comprises four genes, particularly ASCL1, GRP, NOTCH1 and VIM.

In a particular embodiment of the invention the gene panel comprises four genes, particularly NOTCH1, ASCL1, GRP, and VIM.

In a particular embodiment of the invention the gene panel comprises three genes, particularly ASCL1, GRP and NOTCH1.

In a particular embodiment of the invention the gene panel comprises three genes, particularly NOTCH1, ASCL1 and GRP.

In a particular embodiment of the invention the gene panel comprises two genes, particularly GRP and NOTCH1.

In a particular embodiment of the invention the gene panel comprises two genes, particularly NOTCH1 and GRP.

In a particular embodiment of the invention the gene panel comprises one gene, particularly NOTCH1.

In a particular embodiment of the invention the gene panel does not comprise the genes ASCL1 and/or GRP.

In a particular embodiment of the invention the gene panel does not comprise the VIM gene.

In a particular embodiment of the invention the gene panel comprises the ASCL1 gene.

In a particular embodiment of the invention the gene panel comprises the CNN2 gene.

In a particular embodiment of the invention the gene panel comprises the DENND5A gene.

In a particular embodiment of the invention the gene panel comprises the GRP gene.

In a particular embodiment of the invention the gene panel comprises the NOTCH1 gene.

In a particular embodiment of the invention the gene panel comprises the VIM gene.

In a particular embodiment of the invention the gene panel comprises the ZFP36L1 gene.

In a particular embodiment of the invention the gene panel consists of one, two, three, four or five genes.

In a particular embodiment of the invention the gene panel consists of two, three or four genes.

In a particular embodiment of the invention the gene panel comprises one gene.

In a particular embodiment of the invention the up-regulation of CNN2, DENND5A, NOTCH1, VIM, and ZFP36L1 levels after begin of therapy comprising an LSD1 inhibitor is indicative of the response of the patient to the therapy.

In a particular embodiment of the invention the up-regulation of NOTCH1, CNN2, DENND5A, VIM, and ZFP36L1 levels after begin of therapy comprising an LSD1 inhibitor is indicative of the response of the patient to the therapy.

In a particular embodiment of the invention the down-regulation of ASCL1 and GRP levels after begin of therapy comprising an LSD1 inhibitor are indicative of the response of the patient to the therapy.

In a particular embodiment of the invention the gene panel comprises or more genes selected from the group of ASCL1, CNN2, DENND5A, GRP, NOTCH1, VIM, and ZFP36L1, wherein up-regulated levels of CNN2, DENND5A, NOTCH1, VIM, and ZFP36L1 and/or down-regulated levels of ASCL1 and GRP after begin of therapy comprising an LSD1 inhibitor are indicative for a response of the patient to the therapy.

In a particular embodiment of the invention the gene panel comprises or more genes selected from the group of NOTCH1, ASCL1, GRP, CNN2, DENND5A, VIM, and ZFP36L1, wherein up-regulated levels of NOTCH1, CNN2, DENND5A, VIM, and ZFP36L1 and/or down-regulated levels of ASCL1 and GRP after begin of therapy comprising an LSD1 inhibitor are indicative for a response of the patient to the therapy.

In a particular embodiment of the invention the gene panel comprises the ASCL1 gene, wherein down-regulated ASCL1 levels after begin of therapy comprising an LSD1 inhibitor are indicative of the response of the patient to the therapy.

In a particular embodiment of the invention the gene panel comprises the CNN2 gene, wherein up-regulated CNN2 levels after begin of therapy comprising an LSD1 inhibitor are indicative of the response of the patient to the therapy.

In a particular embodiment of the invention the gene panel comprises the DENND5A gene, wherein up-regulated DENND5A levels after begin of therapy comprising an LSD1 inhibitor are indicative of the response of the patient to the therapy.

In a particular embodiment of the invention the gene panel comprises the GRP gene, wherein down-regulated GRP levels after begin of therapy comprising an LSD1 inhibitor are indicative of the response of the patient to the therapy.

In a particular embodiment of the invention the gene panel comprises the NOTCH1 gene, wherein up-regulated NOTCH1 levels after begin of therapy comprising an LSD1 inhibitor are indicative of the response of the patient to the therapy.

In a particular embodiment of the invention the gene panel comprises the VIM gene, wherein up-regulated VIM levels after begin of therapy comprising an LSD1 inhibitor are indicative of the response of the patient to the therapy.

In a particular embodiment of the invention the gene panel comprises the ZFP36L1 gene, wherein up-regulated ZFP36L1 levels after begin of therapy comprising an LSD1 inhibitor are indicative of the response of the patient to the therapy.

In one aspect of the present invention, the LSD1 inhibitor is selected from a compound as described in WO 2011/131697 (A1), WO 2012135113 (A2) and WO 2013/057322 (A1).

In a particular embodiment of the invention the LSD1 inhibitor is selected from the list of:
4-[ [4-[[[(1R,2S)-2-phenylcyclopropyl]amino]methyl]-1-piperidinyl]methyl]-benzoic acid (trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-di amine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine, and a pharmaceutically acceptable salt thereof.

In a particular embodiment of the invention the LSD1 inhibitor is GSK2879552 [CAS Reg. No. 1401966-69-5], also known as 4-[[4-[[[(1R,2S)-2-phenylcyclopropyl]amino]methyl]-1-piperidinyl]methyl]benzoic acid, or a pharmaceutically acceptable salt thereof.

In a particular embodiment of the invention the LSD1 inhibitor is selected from the list of:
(trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-diamine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine, and a pharmaceutically acceptable salt thereof.

In a particular embodiment of the invention the LSD1 inhibitor is (trans)-N1-((1R,2S)-2-phenylcyclopropyl)cyclohexane-1,4-diamine [CAS Reg. No. 1431304-21-0] or a pharmaceutically acceptable salt thereof.

In a particular embodiment of the invention the LSD1 inhibitor is (trans)-N1-((1R,2S)-2-phenylcyclopropyl)cyclohexane-1,4-diamine [CAS Reg. No. 1431304-21-0] or a hydrochloride salt thereof.

In a particular embodiment of the invention the LSD1 inhibitor is (trans)-N1-((1R,2S)-2-phenylcyclopropyl)cyclohexane-1,4-diamine bis-hydrochloride [CAS Reg. No. 1431303-72-8].

In a particular embodiment of the invention the LSD1 inhibitor is administered to a patient in need thereof orally, such as an oral solution.

The mRNA transcript expression levels and/or the expression levels of the translated proteins can either be measured at the site of tumor origin or alternatively derived from the periphery such as whole blood, serum or plasma.

The mRNA transcript expression levels and/or the expression levels of the translated proteins can further be measured from samples like bone marrow, saliva, skin or hair, or from non-primary tumors (e.g. metastases, ascites or circulating tumor cells).

Measurements may be taken from a whole blood specimen, a blood serum specimen, a blood plasma specimen, a bone marrow specimen, or a fresh, frozen or formalin-fixed paraffin embedded primary human tumor specimen.

Measurements may further be taken from saliva specimen, skin specimen or hair specimen, or a fresh, frozen or formalin-fixed paraffin embedded non-primary tumor specimen (e.g. metastases, ascites or circulating tumor cells).

As described above, LSD1 inhibitors have been described for use in the treatment of patients having a neoplastic disease.

In a particular embodiment of the invention the neoplastic disease that is potentially treatable based on the desired LSD1 clinical response is a cancer, particularly a cancer selected from the group consisting of breast cancer, prostate cancer, cervical cancer, ovarian cancer, gastric cancer, colorectal cancer (i.e. including colon cancer and rectal cancer), pancreatic cancer, liver cancer, brain cancer, neuroendocrine cancer, lung cancer, kidney cancer, hematological malignancies, melanoma and sarcomas.

In a particular embodiment of the invention the cancer that is potentially treatable based on the LSD1 response is selected from the group consisting of hematological malignancies, neuroendocrine cancer, breast cancer, cervical cancer, ovarian cancer, colorectal cancer, melanoma and lung cancer.

In a particular embodiment of the invention the neoplastic disease is a cancer selected from the group consisting of blood cancer or lung cancer, more particularly acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

In a particular embodiment of the invention the neoplastic disease is a blood cancer or lung cancer selected from the group of acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

In a particular embodiment of the invention the neoplastic disease is a cancer is selected from the group consisting of acute myeloid leukemia (AML), non-Hodgkin's lymphoma, small cell lung cancer (SCLC), thyroid cancer, and melanoma.

In a particular embodiment of the invention the neoplastic disease is a cancer selected from the group consisting of acute myeloid leukemia (AML), thyroid cancer, melanoma, or small cell lung cancer (SCLC).

In a particular embodiment of the invention the neoplastic disease is a cancer selected from the group consisting of acute myeloid leukemia (AML) and small cell lung cancer (SCLC).

In a particular embodiment of the invention the neoplastic disease is neuroendocrine cancer.

In a particular embodiment of the invention the neoplastic disease is lung cancer.

In a particular embodiment of the invention the neoplastic disease is small cell lung cancer (SCLC).

### DESCRIPTION OF THE DRAWINGS

FIG. 1: ASCL1 as PD biomarker is down-regulated in SCLC cell lines according to Example 1 (RNASeq data, error bars denote 95% confidence interval).

FIG. 2: CNN2 as PD biomarker is up-regulated across cell lines according to Example 1 (RNASeq data, error bars denote 95% confidence interval).

FIG. 3: DENND5A as PD biomarker is up-regulated across cell lines according to Example 1 (RNASeq data, error bars denote 95% confidence interval).

FIG. 4: GRP as PD biomarker is down-regulated in SCLC cell lines according to Example 1 (RNASeq data, error bars denote 95% confidence interval).

FIG. 5: NOTCH1 as PD biomarker is up-regulated across cell lines according to Example 1 (RNASeq data, error bars denote 95% confidence interval).

FIG. 6: VIM as PD biomarker is up-regulated across cell lines according to Example 1 (RNASeq data, error bars denote 95% confidence interval).

FIG. 7: ZFP36L1 as PD biomarker is up-regulated across cell lines according to Example 1 (RNASeq data, error bars denote 95% confidence interval).

FIG. 8: Regulation of candidate PD biomarkers ASCL1 (down) and GRP (down) and NOTCH1 (up) after (trans)-N1-((1R,2S)-2-phenylcyclopropyl)cyclohexane-1,4-diamine (LSD1i) treatment as identified in LSD1i responsive cell lines according to Example 2.

FIG. 9: PD gene expression validated in vivo in SCLC 510A xenografts: ASCL1 transcript down regulation is (trans)-N1-((1R,2S)-2-phenylcyclopropyl)cyclohexane-1,4-diamine (LSD1i) dose and time dependent according to Example 3.

FIG. 10: PD gene expression validated in vivo in SCLC 510A xenografts: GRP transcript down regulation is (trans)-N1-((1R,2S)-2-phenylcyclopropyl)cyclohexane-1,4-diamine (LSD1i) dose and time dependent according to Example 3.

FIG. 11: PD gene expression validated in vivo in SCLC 510A xenografts: NOTCH1 transcript up regulation is (trans)-N1-((1R,2S)-2-phenylcyclopropyl)cyclohexane-1,4-diamine (LSD1i) dose and time dependent according to Example 3.

FIG. 12: Regulation of candidate PD biomarkers ASCL1 (down) and NOTCH1 (up) after (trans)-N1-((1R,2S)-2-phenylcyclopropyl)cyclohexane-1,4-diamine (LSD1i) treatment as identified in LSD1i treated PDX cultures according to Example 2.

FIG. 13: PD gene expression validated in vivo in SCLC FHSC04 PDX: NOTCH1 transcript up regulation; ASCL1 and GRP down regulation upon exposure to (trans)-N1-((1R,2S)-2-phenylcyclopropyl)cyclohexane-1,4-diamine (LSD1i) according to Example 3.

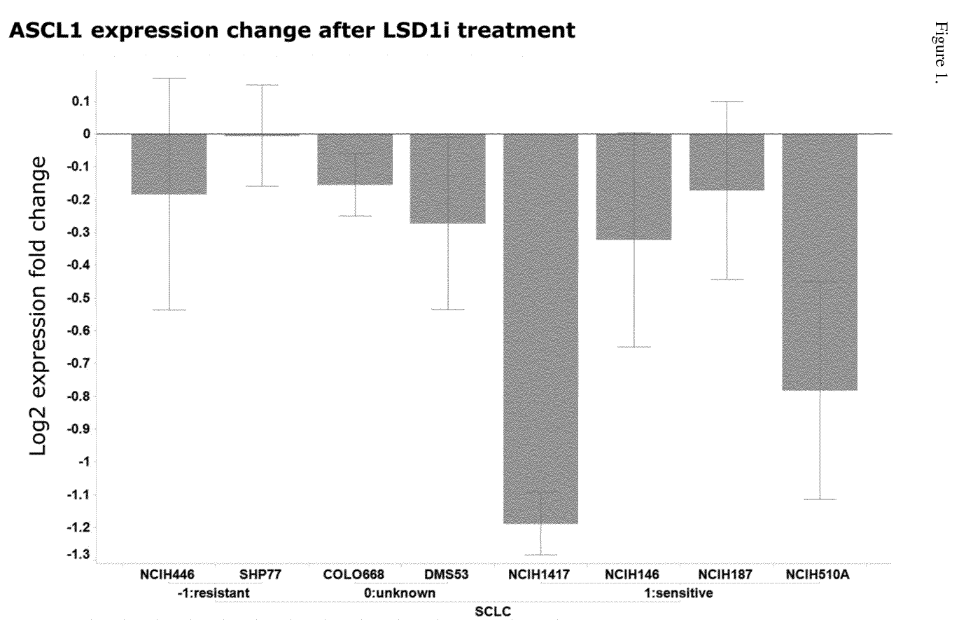

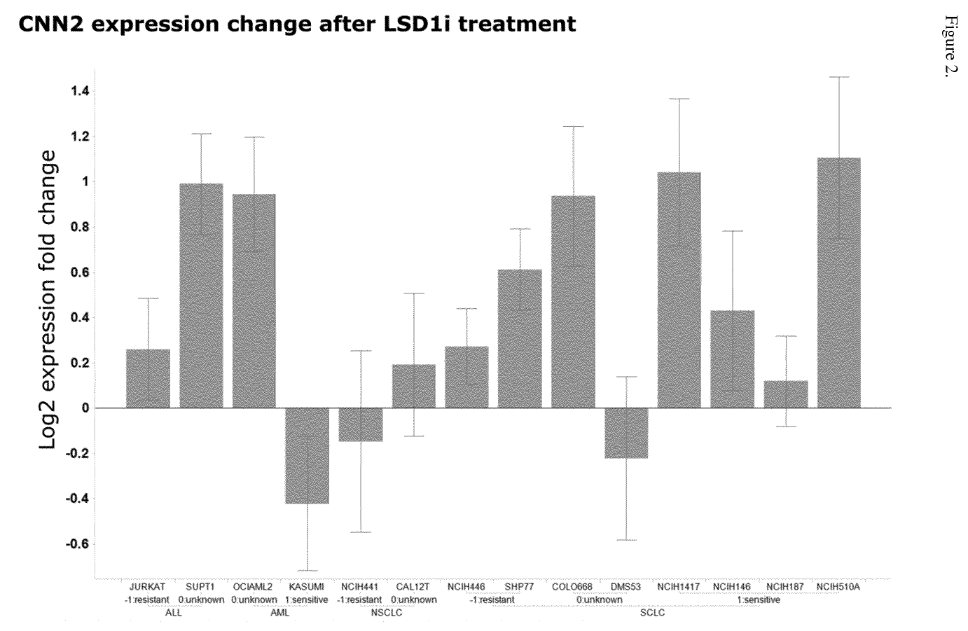

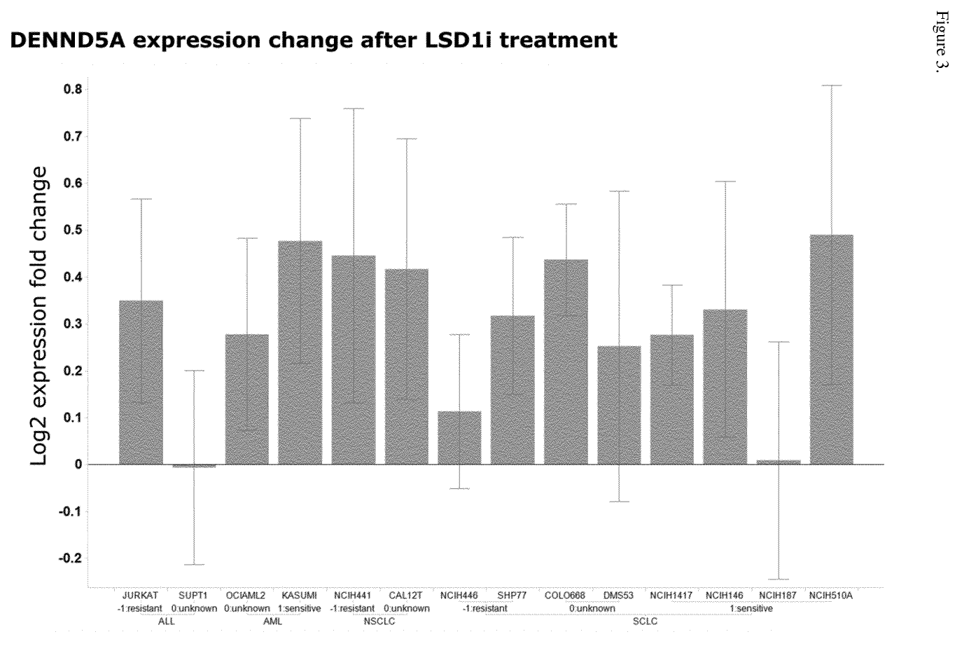

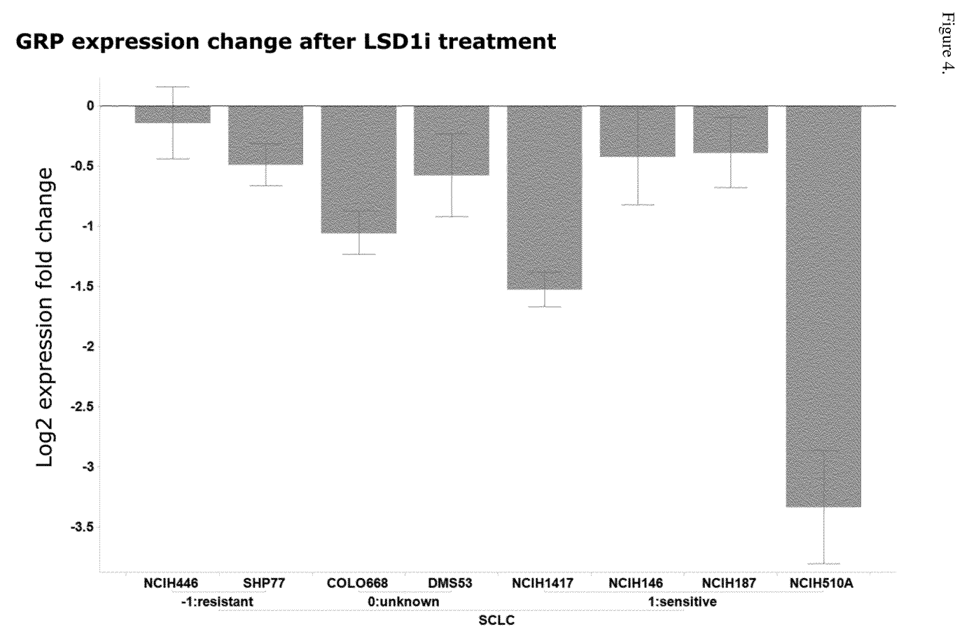

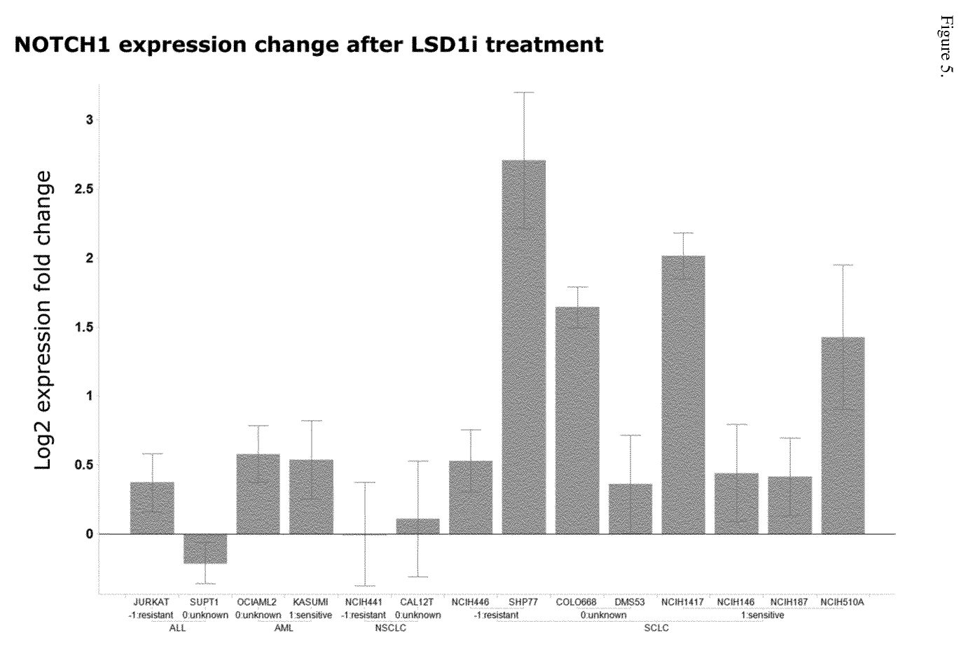

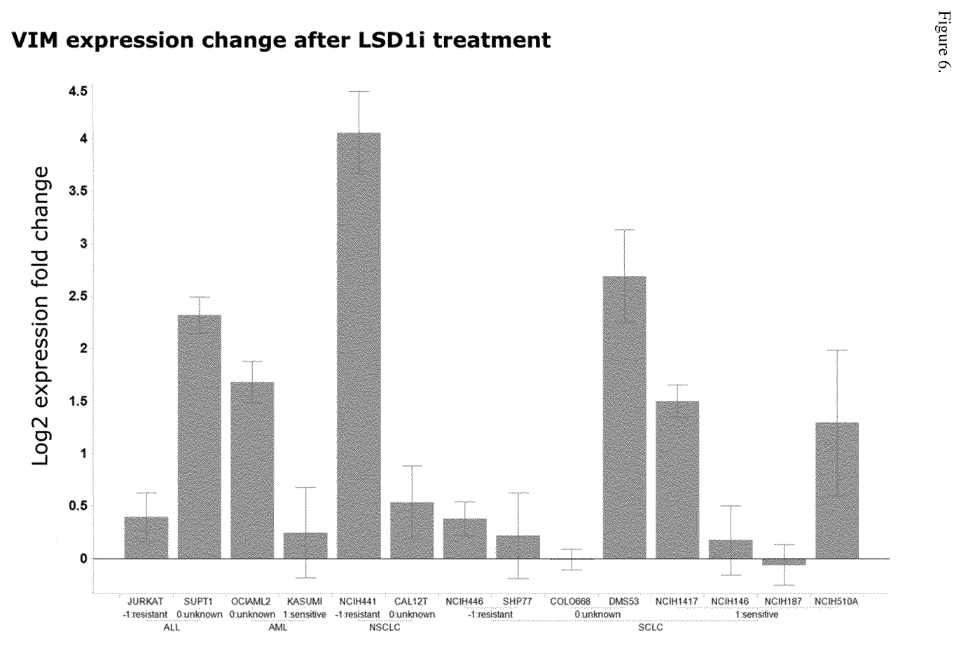

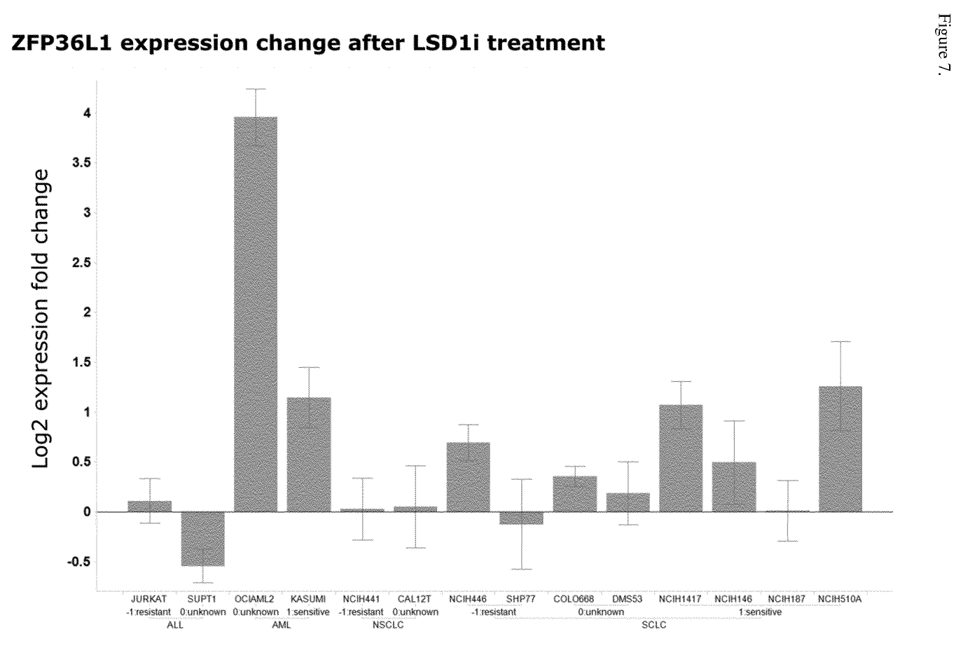

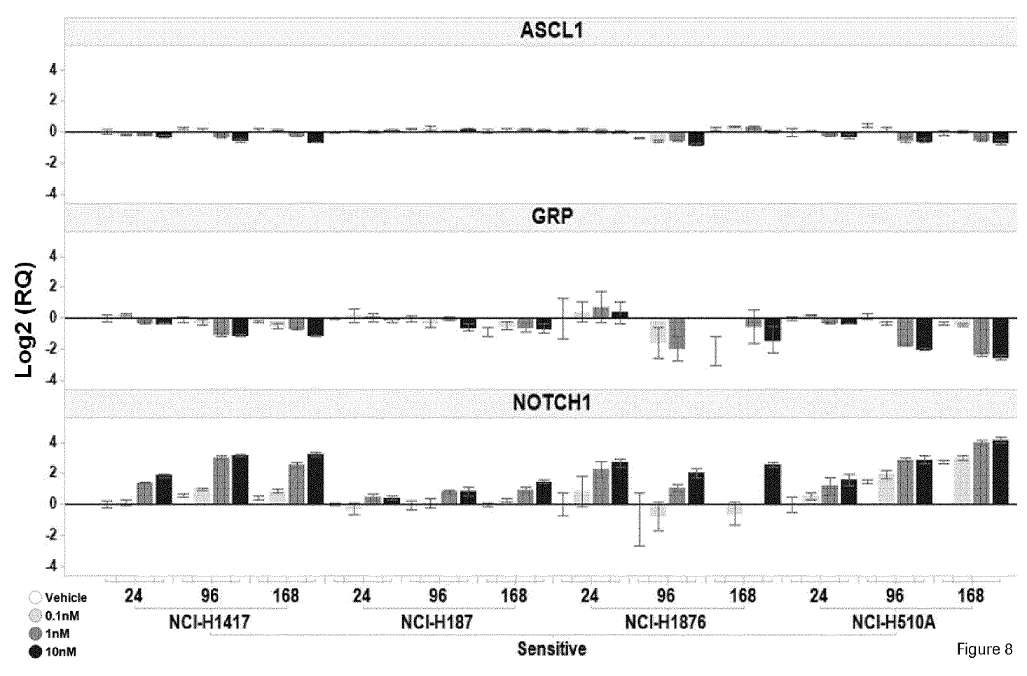

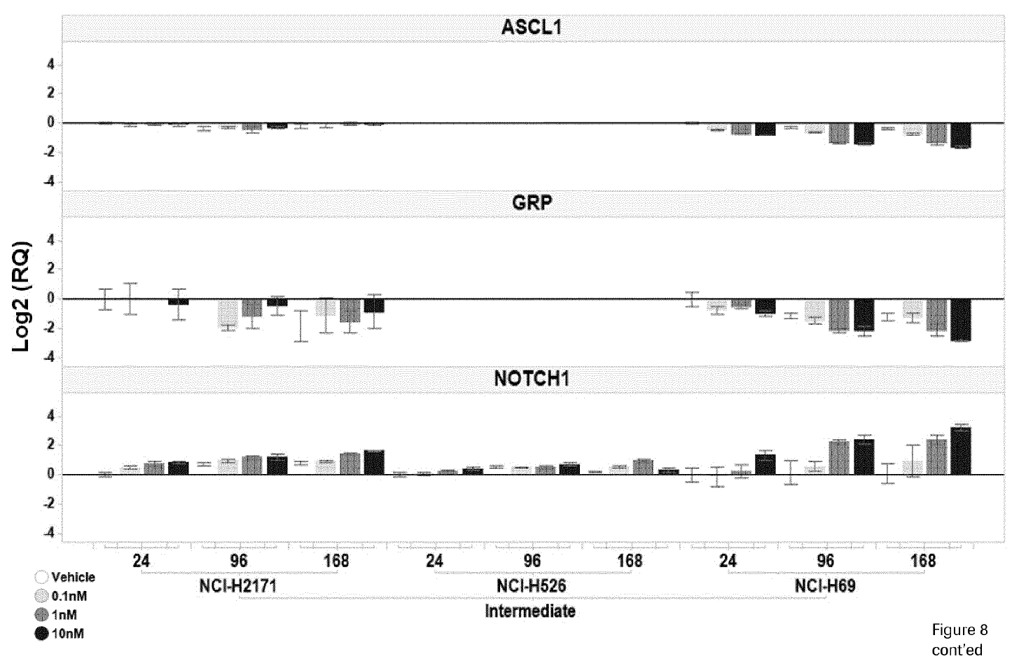

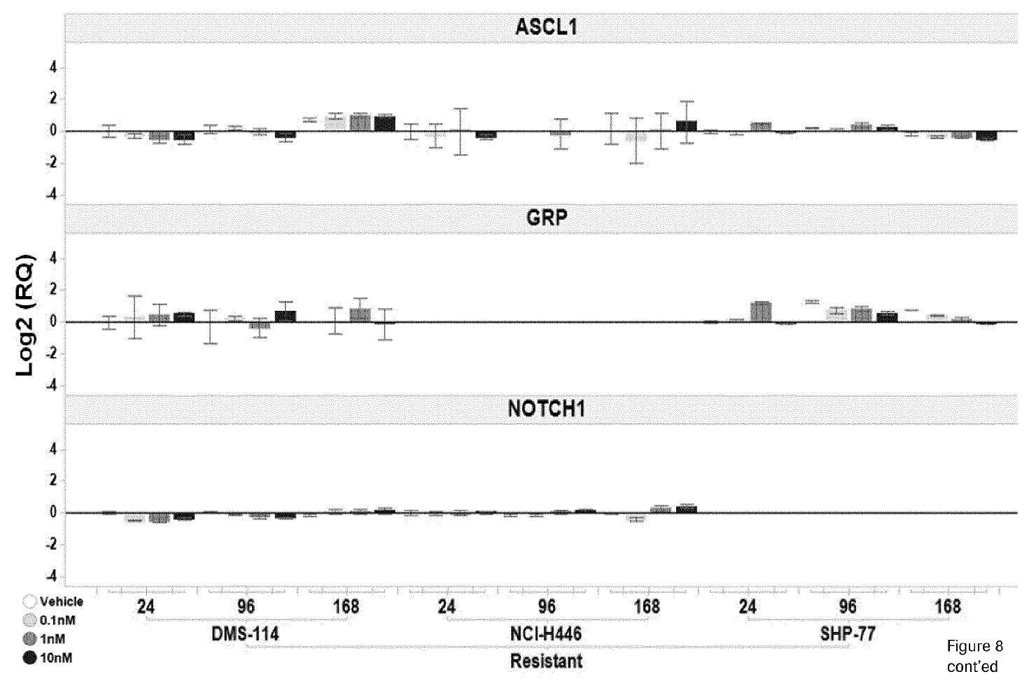

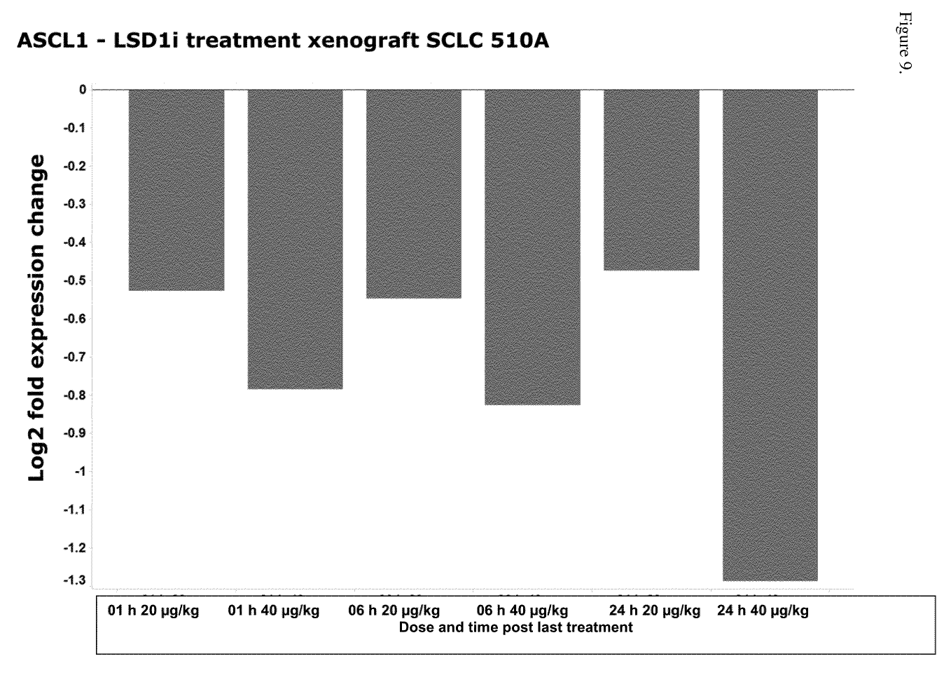

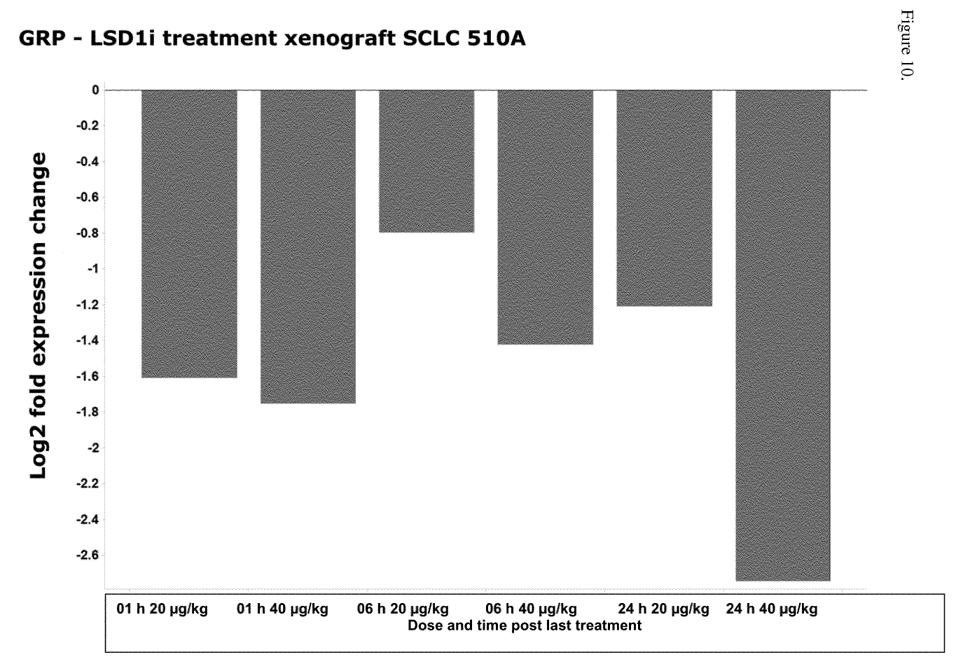

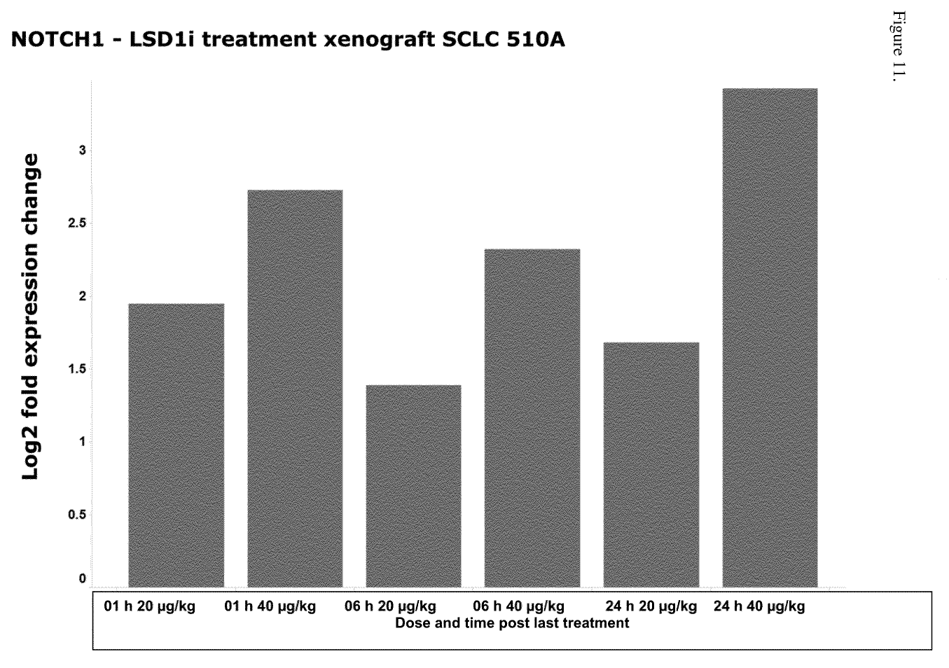

### EXAMPLES

The following examples 1 to 3 are provided for illustration of the invention. They should not be considered as limiting the scope of the invention, but merely as being representative thereof.

### Example 1. Significant Expression Change of PD Markers in Multiple Cell Lines

A panel of 14 cell lines was treated with (trans)-N1-((1R,2S)-2-phenylcyclopropyl)cyclohexane-1,4-diamine (5 nM) or control vehicle for 6 days. The panel included eight small cell lung cancer cell lines (COLO 668, DMS 53, NCI-H146, NCI-H187, NCI-H446, NCI-H510A, NCI-H1417, SHP-77), two non-small-cell lung cancer cell lines (CAL-12T, NCI-H441), two acute myelogenic leukemia cell lines (KASUMI, OCI-AML2), and two acute lymphoblastic T-cell leukemia cell lines (JURKAT, SUP-T1).

Each cell line-treatment pair contained two to four biological replicates. Table 3 lists all cell lines, disease type, treatment, and number of replicates in the study.

TABLE 3
Cell lines and number of biological replicates in the panel. RG6016
is (trans)-N1-((1R,2S)-2-phenylcyclopropyl)cyclohexane-1,4-diamine

| | | | Biological |
|---|---|---|---|
| Cell line | Disease | Treatment | replicates |
| COLO 668 | Small cell lung cancer | RG6016 | 3 |
| | | Vehicle | 3 |
| DMS 53 | Small cell lung cancer | RG6016 | 3 |
| | | Vehicle | 3 |
| NCI-H146 | Small cell lung cancer | RG6016 | 4 |
| | | Vehicle | 2 |
| NCI-H187 | Small cell lung cancer | RG6016 | 4 |
| | | Vehicle | 2 |
| NCI-H446 | Small cell lung cancer | RG6016 | 3 |
| | | Vehicle | 3 |
| NCI-H510A | Small cell lung cancer | RG6016 | 3 |
| | | Vehicle | 3 |
| NCI-H1417 | Small cell lung cancer | RG6016 | 3 |
| | | Vehicle | 3 |
| SHP-77 | Small cell lung cancer | RG6016 | 3 |
| | | Vehicle | 3 |
| CAL-12T | Non-small-cell lung caner | RG6016 | 2 |
| | | Vehicle | 2 |
| NCI-H441 | Non-small-cell lung cancer | RG6016 | 3 |
| | | Vehicle | 2 |
| KASUMI | Acute myelogenic leukemia | RG6016 | 3 |
| | | Vehicle | 3 |
| OCI-AML2 | Acute myelogenic leukemia | RG6016 | 3 |
| | | Vehicle | 3 |
| JURKAT | Acute lymphoblastic T-cell | RG6016 | 3 |
| | leukemia | Vehicle | 3 |
| SUP-T1 | Acute lymphoblastic T-cell | RG6016 | 3 |
| | leukemia | Vehicle | 3 |

Expression data were obtained from whole transcriptomic RNA sequencing (RNA-seq) by Illumina, Inc. (San Diego, Calif.). The Illumina HiSeq machine generates raw base calls in reads of 50 or 100 bp length, which are subjected to several data analysis steps. The RNA-seq is conducted at 40 to 50 million reads per sample. This number provides relatively high sensitivity to detect low-expressed genes while allowing for cost-effective multiplexing of samples. RNA is prepared by standard kits and RNA libraries by polyA TruSeq Illumina kits. 100 ng of mRNA per cell line is used for each RNA-seq reaction. A number of quality control procedures are applied to the RNA-seq data for each sample. The Illumina HiSeq software reports the total number of clusters (DNA fragments) loaded in each lane, percent passing sequencing quality filters (which identifies errors due to overloading and sequencing chemistry), a phred quality score for each base of each sequence read, overall average phred scores for each sequencing cycle, and overall percent error (based on alignment to the reference genome). For each RNA-seq sample, the percentage of reads that contain mitochondrial and ribosomal RNA was calculated. The FASTQC package was used to provide additional QC metrics (base distribution, sequence duplication, overrepresented sequences, and enriched kmers) and a graphical summary. Raw reads were aligned against the human genome (hg19) using GSNAP and recommended options for RNASeq data. In addition to the genome sequence, GSNAP is given a database of human splice junctions and transcripts based on Ensembl v73. Resulting SAM files are then converted to sorted BAM files using Samtools. Gene expression values are calculated both as RPKM values following (Mortazavi A. et al. (2008) Nature Methods 5:621-628) and as read counts.

Differential gene expression analysis was performed using the DESeq2 package (Love M. I. et al. (2014) Genome Biology 15:550). Read counts from RNA-seq data were analyzed via a multi-factor generalized linear model of the negative binomial family, considering both treatment and the cell line type as factors that explain the changes in gene expression values.

The following filtering process was applied to identify genes that can serve as PD markers in surrogate tissues (skin or blood):
1. The gene was significantly differentially expressed in SCLC cell lines with Benjamini-Hochberg-adjusted P value<0.05, as determined by DESeq2
2. The gene had a median fold change greater than 1.2 after the treatment with (trans)-N1-((1R,2S)-2-phenylcyclopropyl)cyclohexane-1,4-diamine across all cell lines.
3. The minimal expression level of the gene in normal blood and skin tissues was greater than one read per kilobase per million reads (RPKM, Mortazavi et al.), as obtained from the Genotype-Tissue Experssion (GTEx) project (The GTEx Consortium (2015) Science 348(6235):648-660).
4. The minimal expression level of the gene in PBMC tissues was greater than 20 fragments per kilobase per million (FPKM; Trapnell C. et al. (2010) Nature Biotechnology 28:511-515) as obtained from the Ohmomo et al. study (Ohmomo H. et al. (2014) PLOS ONE 9(8):1-11).
5. The gene is a target of LSD1, as determined by chromatin immunoprecipitation (ChIP) assay from the Adamo et al. study, or interacts with the LSD1 gene, as annotated by the BioGRID database (thebiogrid.org).

Table 4 contains the 5 genes that satisfy all the filtering criteria.

TABLE 4
Candidate up-regulated PD markers.

| | | | | PBMC | |
|---|---|---|---|---|---|
| | Median | Blood exp | Skin exp | exp | SCLC |
| Gene | Log2 FC | (RPKM) | (RPKM) | (FPKM) | Adj P value |
| NOTCH1 | 0.49 | 1.60 | 1.02 | 22.48 | 9.49E−08 |
| DENND5A | 0.32 | 1.80 | 2.24 | 23.92 | 5.26E−08 |
| CNN2 | 0.35 | 20.18 | 15.82 | 137.48 | 2.08E−05 |
| ZFP36L1 | 0.27 | 7.35 | 25.74 | 111.55 | 3.41E−04 |
| VIM | 0.46 | 3.85 | 62.69 | 578.42 | 7.78E−03 |

Additionally, we analyzed differential gene expression for a set of 35 genes which were previously identified as predictive biomarkers of response to LSD1 treatment. Out of this set of genes, the two neuroendocrine markers ASCL1 and GRP were also included as candidate PD markers, because they were very significantly down-regulated after (trans)-N1-((1R,2S)-2-phenylcyclopropyl)cyclohexane-1,4-diamine treatment (Table 5).

TABLE 5
Down-regulation of ASCL1 and GRP after (trans)-N1-((1R,2S)-2-
phenylcyclopropyl)cyclohexane-1,4-diamine treatment

| | | SCLC |
|---|---|---|
| Gene | Median Log2 FC | Adj P value |
| ASCL1 | −0.16 | 3.53E−05 |
| GRP | −0.26 | 7.56E−07 |

Differentially expressed pathway analysis was done by the MetaBase R Library (Thompson Reuters). The algorithm calculates the distances between samples in the gene expression space in the pathway, and tests whether the distances between samples in the same experiment group is significantly smaller than distances between samples in different experiment groups. The pathway analysis identified development NOTCH signaling pathway as one of the most significantly differentially expressed pathway after the (trans)-N1-((1R,2S)-2-phenylcyclopropyl)cyclohexane-1,4-diamine treatment.

### Example 2. Time- and Dosage-Dependent Expression Change of Candidate PD Markers in SCLC Cell Lines

Seven small cell lung cancer cell lines were treated with 0.1 nM, 1 nM and 10 nM (trans)-N1-((1R,2S)-2-phenylcyclopropyl)cyclohexane-1,4-diamine or vehicle 24 hr, 96 hr and 7 days. Lysates were prepared by lysis in RNA lysis buffer containing 1% β-mercaptoethanol. RNA extraction was performed using a Maxwell 16 instrument and a Maxwell 16 LEV simply RNA cells Kit (Promega), according to the manufacturer's instructions. The mRNA expression levels of each cell line after treatment were then measured using qRT-PCR. The RT-qPCR reaction was conducted using a one-step kit (ABI), with a duplexed house-keeping control (Assay ID Hs02800695_m1) using the assays in the table below. The log 2 fold changes for each gene were calculated by comparing with the vehicle controlled samples at 24 hr sampling point.

Table 6 contains the dose-dependent effect on expression changes for candidate PD markers ASCL1 (Assay ID Hs04187546_g1), GRP (Assay ID Hs01107047_m1) and NOTCH1 (Assay ID Hs01062014_m1).

TABLE 6
Dose-dependent expression change for candidate PD markers in SCLC cell lines.

| Cell line | Dose | Concentration | Time (hrs) | Target | Log2_FC | Log2_Error |
|---|---|---|---|---|---|---|
| DMS-114 | Vehicle | Vehicle | 24 | ASCL1 | 0.00 | 0.38 |
| DMS-114 | Vehicle | Vehicle | 96 | ASCL1 | 0.10 | 0.27 |
| DMS-114 | Vehicle | Vehicle | 168 | ASCL1 | 0.72 | 0.14 |
| DMS-114 | Medium | 1 nM | 24 | ASCL1 | −0.50 | 0.21 |
| DMS-114 | Medium | 1 nM | 96 | ASCL1 | −0.04 | 0.19 |
| DMS-114 | Medium | 1 nM | 168 | ASCL1 | 1.02 | 0.09 |
| DMS-114 | High | 10 nM | 24 | ASCL1 | −0.52 | 0.29 |
| DMS-114 | High | 10 nM | 96 | ASCL1 | −0.38 | 0.25 |
| DMS-114 | High | 10 nM | 168 | ASCL1 | 0.94 | 0.14 |
| DMS-114 | Low | 0.1 nM | 24 | ASCL1 | −0.28 | 0.15 |
| DMS-114 | Low | 0.1 nM | 96 | ASCL1 | 0.18 | 0.10 |
| DMS-114 | Low | 0.1 nM | 168 | ASCL1 | 0.93 | 0.19 |
| DMS-114 | Vehicle | Vehicle | 24 | GRP | 0.00 | 0.42 |
| DMS-114 | Vehicle | Vehicle | 96 | GRP | −0.28 | 1.02 |
| DMS-114 | Vehicle | Vehicle | 168 | GRP | ND | ND |
| DMS-114 | Medium | 1 nM | 24 | GRP | 0.45 | 0.66 |
| DMS-114 | Medium | 1 nM | 96 | GRP | −0.34 | 0.58 |
| DMS-114 | Medium | 1 nM | 168 | GRP | 0.86 | 0.63 |
| DMS-114 | High | 10 nM | 24 | GRP | 0.51 | 0.09 |
| DMS-114 | High | 10 nM | 96 | GRP | 0.67 | 0.58 |
| DMS-114 | High | 10 nM | 168 | GRP | −0.12 | 0.97 |
| DMS-114 | Low | 0.1 nM | 24 | GRP | 0.32 | 1.32 |
| DMS-114 | Low | 0.1 nM | 96 | GRP | 0.25 | 0.16 |
| DMS-114 | Low | 0.1 nM | 168 | GRP | 0.08 | 0.84 |
| DMS-114 | Vehicle | Vehicle | 24 | NOTCH1 | 0.00 | 0.08 |
| DMS-114 | Vehicle | Vehicle | 96 | NOTCH1 | 0.03 | 0.04 |
| DMS-114 | Vehicle | Vehicle | 168 | NOTCH1 | −0.12 | 0.06 |
| DMS-114 | Medium | 1 nM | 24 | NOTCH1 | −0.47 | 0.11 |
| DMS-114 | Medium | 1 nM | 96 | NOTCH1 | −0.23 | 0.09 |
| DMS-114 | Medium | 1 nM | 168 | NOTCH1 | 0.08 | 0.13 |
| DMS-114 | High | 10 nM | 24 | NOTCH1 | −0.35 | 0.07 |
| DMS-114 | High | 10 nM | 96 | NOTCH1 | −0.30 | 0.05 |
| DMS-114 | High | 10 nM | 168 | NOTCH1 | 0.13 | 0.18 |
| DMS-114 | Low | 0.1 nM | 24 | NOTCH1 | −0.47 | 0.07 |
| DMS-114 | Low | 0.1 nM | 96 | NOTCH1 | −0.11 | 0.05 |
| DMS-114 | Low | 0.1 nM | 168 | NOTCH1 | 0.08 | 0.14 |
| NCI-H1417 | Vehicle | Vehicle | 24 | ASCL1 | 0.00 | 0.16 |
| NCI-H1417 | Vehicle | Vehicle | 96 | ASCL1 | 0.21 | 0.07 |
| NCI-H1417 | Vehicle | Vehicle | 168 | ASCL1 | 0.14 | 0.09 |
| NCI-H1417 | Medium | 1 nM | 24 | ASCL1 | −0.20 | 0.04 |
| NCI-H1417 | Medium | 1 nM | 96 | ASCL1 | −0.27 | 0.12 |
| NCI-H1417 | Medium | 1 nM | 168 | ASCL1 | −0.19 | 0.11 |
| NCI-H1417 | High | 10 nM | 24 | ASCL1 | −0.25 | 0.07 |
| NCI-H1417 | High | 10 nM | 96 | ASCL1 | −0.54 | 0.10 |
| NCI-H1417 | High | 10 nM | 168 | ASCL1 | −0.66 | 0.03 |
| NCI-H1417 | Low | 0.1 nM | 24 | ASCL1 | −0.19 | 0.04 |
| NCI-H1417 | Low | 0.1 nM | 96 | ASCL1 | 0.12 | 0.10 |
| NCI-H1417 | Low | 0.1 nM | 168 | ASCL1 | 0.13 | 0.06 |
| NCI-H1417 | Vehicle | Vehicle | 24 | GRP | 0.00 | 0.23 |
| NCI-H1417 | Vehicle | Vehicle | 96 | GRP | −0.12 | 0.17 |
| NCI-H1417 | Vehicle | Vehicle | 168 | GRP | −0.22 | 0.09 |
| NCI-H1417 | Medium | 1 nM | 24 | GRP | −0.25 | 0.09 |
| NCI-H1417 | Medium | 1 nM | 96 | GRP | −1.06 | 0.12 |
| NCI-H1417 | Medium | 1 nM | 168 | GRP | −0.68 | 0.08 |
| NCI-H1417 | High | 10 nM | 24 | GRP | −0.35 | 0.04 |
| NCI-H1417 | High | 10 nM | 96 | GRP | −1.13 | 0.09 |
| NCI-H1417 | High | 10 nM | 168 | GRP | −1.14 | 0.06 |
| NCI-H1417 | Low | 0.1 nM | 24 | GRP | 0.24 | 0.06 |
| NCI-H1417 | Low | 0.1 nM | 96 | GRP | −0.25 | 0.19 |
| NCI-H1417 | Low | 0.1 nM | 168 | GRP | −0.51 | 0.14 |
| NCI-H1417 | Vehicle | Vehicle | 24 | NOTCH1 | 0.00 | 0.23 |
| NCI-H1417 | Vehicle | Vehicle | 96 | NOTCH1 | 0.58 | 0.12 |
| NCI-H1417 | Vehicle | Vehicle | 168 | NOTCH1 | 0.42 | 0.14 |
| NCI-H1417 | Medium | 1 nM | 24 | NOTCH1 | 1.40 | 0.05 |
| NCI-H1417 | Medium | 1 nM | 96 | NOTCH1 | 3.04 | 0.09 |
| NCI-H1417 | Medium | 1 nM | 168 | NOTCH1 | 2.55 | 0.13 |
| NCI-H1417 | High | 10 nM | 24 | NOTCH1 | 1.86 | 0.12 |
| NCI-H1417 | High | 10 nM | 96 | NOTCH1 | 3.14 | 0.09 |
| NCI-H1417 | High | 10 nM | 168 | NOTCH1 | 3.22 | 0.12 |
| NCI-H1417 | Low | 0.1 nM | 24 | NOTCH1 | 0.14 | 0.18 |
| NCI-H1417 | Low | 0.1 nM | 96 | NOTCH1 | 0.98 | 0.06 |
| NCI-H1417 | Low | 0.1 nM | 168 | NOTCH1 | 0.86 | 0.11 |
| NCI-H187 | Vehicle | Vehicle | 24 | ASCL1 | 0.00 | 0.04 |
| NCI-H187 | Vehicle | Vehicle | 96 | ASCL1 | 0.23 | 0.04 |
| NCI-H187 | Vehicle | Vehicle | 168 | ASCL1 | 0.05 | 0.11 |
| NCI-H187 | Medium | 1 nM | 24 | ASCL1 | −0.01 | 0.06 |
| NCI-H187 | Medium | 1 nM | 96 | ASCL1 | 0.05 | 0.03 |
| NCI-H187 | Medium | 1 nM | 168 | ASCL1 | 0.16 | 0.07 |
| NCI-H187 | High | 10 nM | 24 | ASCL1 | 0.13 | 0.04 |
| NCI-H187 | High | 10 nM | 96 | ASCL1 | 0.13 | 0.08 |
| NCI-H187 | High | 10 nM | 168 | ASCL1 | 0.07 | 0.06 |
| NCI-H187 | Low | 0.1 nM | 24 | ASCL1 | 0.07 | 0.05 |
| NCI-H187 | Low | 0.1 nM | 96 | ASCL1 | 0.24 | 0.14 |
| NCI-H187 | Low | 0.1 nM | 168 | ASCL1 | 0.11 | 0.09 |
| NCI-H187 | Vehicle | Vehicle | 24 | GRP | 0.00 | 0.03 |
| NCI-H187 | Vehicle | Vehicle | 96 | GRP | −0.03 | 0.16 |
| NCI-H187 | Vehicle | Vehicle | 168 | GRP | −0.88 | 0.29 |
| NCI-H187 | Medium | 1 nM | 24 | GRP | 0.05 | 0.24 |
| NCI-H187 | Medium | 1 nM | 96 | GRP | −0.03 | 0.10 |
| NCI-H187 | Medium | 1 nM | 168 | GRP | −0.59 | 0.31 |
| NCI-H187 | High | 10 nM | 24 | GRP | −0.16 | 0.13 |
| NCI-H187 | High | 10 nM | 96 | GRP | −0.61 | 0.22 |
| NCI-H187 | High | 10 nM | 168 | GRP | −0.67 | 0.29 |
| NCI-H187 | Low | 0.1 nM | 24 | GRP | 0.16 | 0.44 |
| NCI-H187 | Low | 0.1 nM | 96 | GRP | −0.29 | 0.31 |
| NCI-H187 | Low | 0.1 nM | 168 | GRP | −0.50 | 0.27 |
| NCI-H187 | Vehicle | Vehicle | 24 | NOTCH1 | 0.00 | 0.08 |
| NCI-H187 | Vehicle | Vehicle | 96 | NOTCH1 | −0.06 | 0.32 |
| NCI-H187 | Vehicle | Vehicle | 168 | NOTCH1 | −0.02 | 0.13 |
| NCI-H187 | Medium | 1 nM | 24 | NOTCH1 | 0.49 | 0.23 |
| NCI-H187 | Medium | 1 nM | 96 | NOTCH1 | 0.81 | 0.12 |
| NCI-H187 | Medium | 1 nM | 168 | NOTCH1 | 0.95 | 0.16 |
| NCI-H187 | High | 10 nM | 24 | NOTCH1 | 0.42 | 0.10 |
| NCI-H187 | High | 10 nM | 96 | NOTCH1 | 0.83 | 0.28 |
| NCI-H187 | High | 10 nM | 168 | NOTCH1 | 1.44 | 0.11 |
| NCI-H187 | Low | 0.1 nM | 24 | NOTCH1 | −0.27 | 0.39 |
| NCI-H187 | Low | 0.1 nM | 96 | NOTCH1 | 0.10 | 0.28 |
| NCI-H187 | Low | 0.1 nM | 168 | NOTCH1 | 0.27 | 0.09 |
| NCI-H1876 | Vehicle | Vehicle | 24 | ASCL1 | 0.00 | 0.07 |
| NCI-H1876 | Vehicle | Vehicle | 96 | ASCL1 | −0.42 | 0.03 |
| NCI-H1876 | Vehicle | Vehicle | 168 | ASCL1 | 0.17 | 0.11 |
| NCI-H1876 | Medium | 1 nM | 24 | ASCL1 | 0.04 | 0.09 |
| NCI-H1876 | Medium | 1 nM | 96 | ASCL1 | −0.54 | 0.04 |
| NCI-H1876 | Medium | 1 nM | 168 | ASCL1 | 0.32 | 0.06 |
| NCI-H1876 | High | 10 nM | 24 | ASCL1 | −0.02 | 0.08 |
| NCI-H1876 | High | 10 nM | 96 | ASCL1 | −0.82 | 0.06 |
| NCI-H1876 | High | 10 nM | 168 | ASCL1 | 0.03 | 0.07 |
| NCI-H1876 | Low | 0.1 nM | 24 | ASCL1 | 0.16 | 0.07 |
| NCI-H1876 | Low | 0.1 nM | 96 | ASCL1 | −0.58 | 0.08 |
| NCI-H1876 | Low | 0.1 nM | 168 | ASCL1 | 0.35 | 0.07 |
| NCI-H1876 | Vehicle | Vehicle | 24 | GRP | 0.00 | 1.32 |
| NCI-H1876 | Vehicle | Vehicle | 96 | GRP | ND | ND |
| NCI-H1876 | Vehicle | Vehicle | 168 | GRP | −2.12 | 0.93 |
| NCI-H1876 | Medium | 1 nM | 24 | GRP | 0.70 | 1.00 |
| NCI-H1876 | Medium | 1 nM | 96 | GRP | −1.96 | 0.79 |
| NCI-H1876 | Medium | 1 nM | 168 | GRP | −0.54 | 1.07 |
| NCI-H1876 | High | 10 nM | 24 | GRP | 0.38 | 0.71 |
| NCI-H1876 | High | 10 nM | 96 | GRP | ND | ND |
| NCI-H1876 | High | 10 nM | 168 | GRP | −1.38 | 0.88 |
| NCI-H1876 | Low | 0.1 nM | 24 | GRP | 0.41 | 0.65 |
| NCI-H1876 | Low | 0.1 nM | 96 | GRP | −1.58 | 1.04 |
| NCI-H1876 | Low | 0.1 nM | 168 | GRP | ND | ND |
| NCI-H1876 | Vehicle | Vehicle | 24 | NOTCH1 | 0.00 | 0.74 |
| NCI-H1876 | Vehicle | Vehicle | 96 | NOTCH1 | −0.96 | 1.74 |
| NCI-H1876 | Vehicle | Vehicle | 168 | NOTCH1 | ND | ND |
| NCI-H1876 | Medium | 1 nM | 24 | NOTCH1 | 2.28 | 0.52 |
| NCI-H1876 | Medium | 1 nM | 96 | NOTCH1 | 1.08 | 0.24 |
| NCI-H1876 | Medium | 1 nM | 168 | NOTCH1 | ND | ND |
| NCI-H1876 | High | 10 nM | 24 | NOTCH1 | 2.67 | 0.27 |
| NCI-H1876 | High | 10 nM | 96 | NOTCH1 | 2.07 | 0.30 |
| NCI-H1876 | High | 10 nM | 168 | NOTCH1 | 2.55 | 0.16 |
| NCI-H1876 | Low | 0.1 nM | 24 | NOTCH1 | 0.82 | 0.99 |
| NCI-H1876 | Low | 0.1 nM | 96 | NOTCH1 | −0.76 | 0.91 |
| NCI-H1876 | Low | 0.1 nM | 168 | NOTCH1 | −0.59 | 0.76 |
| NCI-H2171 | Vehicle | Vehicle | 24 | ASCL1 | 0.00 | 0.07 |
| NCI-H2171 | Vehicle | Vehicle | 96 | ASCL1 | −0.36 | 0.12 |
| NCI-H2171 | Vehicle | Vehicle | 168 | ASCL1 | −0.18 | 0.15 |
| NCI-H2171 | Medium | 1 nM | 24 | ASCL1 | −0.06 | 0.10 |
| NCI-H2171 | Medium | 1 nM | 96 | ASCL1 | −0.43 | 0.21 |
| NCI-H2171 | Medium | 1 nM | 168 | ASCL1 | −0.02 | 0.09 |
| NCI-H2171 | High | 10 nM | 24 | ASCL1 | −0.16 | 0.07 |
| NCI-H2171 | High | 10 nM | 96 | ASCL1 | −0.27 | 0.08 |
| NCI-H2171 | High | 10 nM | 168 | ASCL1 | −0.11 | 0.03 |
| NCI-H2171 | Low | 0.1 nM | 24 | ASCL1 | −0.14 | 0.08 |
| NCI-H2171 | Low | 0.1 nM | 96 | ASCL1 | −0.29 | 0.09 |
| NCI-H2171 | Low | 0.1 nM | 168 | ASCL1 | −0.16 | 0.15 |
| NCI-H2171 | Vehicle | Vehicle | 24 | GRP | 0.00 | 0.71 |
| NCI-H2171 | Vehicle | Vehicle | 96 | GRP | ND | ND |
| NCI-H2171 | Vehicle | Vehicle | 168 | GRP | −1.84 | 1.06 |
| NCI-H2171 | Medium | 1 nM | 24 | GRP | ND | ND |
| NCI-H2171 | Medium | 1 nM | 96 | GRP | −1.17 | 0.84 |
| NCI-H2171 | Medium | 1 nM | 168 | GRP | −1.56 | 0.73 |
| NCI-H2171 | High | 10 nM | 24 | GRP | −0.37 | 1.06 |
| NCI-H2171 | High | 10 nM | 96 | GRP | −0.46 | 0.66 |
| NCI-H2171 | High | 10 nM | 168 | GRP | −0.87 | 1.17 |
| NCI-H2171 | Low | 0.1 nM | 24 | GRP | 0.01 | 1.05 |
| NCI-H2171 | Low | 0.1 nM | 96 | GRP | −1.96 | 0.17 |
| NCI-H2171 | Low | 0.1 nM | 168 | GRP | −1.11 | 1.23 |
| NCI-H2171 | Vehicle | Vehicle | 24 | NOTCH1 | 0.00 | 0.14 |
| NCI-H2171 | Vehicle | Vehicle | 96 | NOTCH1 | 0.73 | 0.09 |
| NCI-H2171 | Vehicle | Vehicle | 168 | NOTCH1 | 0.79 | 0.12 |
| NCI-H2171 | Medium | 1 nM | 24 | NOTCH1 | 0.75 | 0.13 |
| NCI-H2171 | Medium | 1 nM | 96 | NOTCH1 | 1.17 | 0.08 |
| NCI-H2171 | Medium | 1 nM | 168 | NOTCH1 | 1.41 | 0.07 |
| NCI-H2171 | High | 10 nM | 24 | NOTCH1 | 0.82 | 0.07 |
| NCI-H2171 | High | 10 nM | 96 | NOTCH1 | 1.22 | 0.20 |
| NCI-H2171 | High | 10 nM | 168 | NOTCH1 | 1.63 | 0.06 |
| NCI-H2171 | Low | 0.1 nM | 24 | NOTCH1 | 0.49 | 0.10 |
| NCI-H2171 | Low | 0.1 nM | 96 | NOTCH1 | 0.95 | 0.11 |
| NCI-H2171 | Low | 0.1 nM | 168 | NOTCH1 | 0.90 | 0.06 |
| NCI-H446 | Vehicle | Vehicle | 24 | ASCL1 | 0.00 | 0.48 |
| NCI-H446 | Vehicle | Vehicle | 96 | ASCL1 | ND | ND |
| NCI-H446 | Vehicle | Vehicle | 168 | ASCL1 | 0.16 | 0.99 |
| NCI-H446 | Medium | 1 nM | 24 | ASCL1 | 0.00 | 1.45 |
| NCI-H446 | Medium | 1 nM | 96 | ASCL1 | −0.20 | 0.93 |
| NCI-H446 | Medium | 1 nM | 168 | ASCL1 | 0.02 | 1.12 |
| NCI-H446 | High | 10 nM | 24 | ASCL1 | −0.34 | 0.16 |
| NCI-H446 | High | 10 nM | 96 | ASCL1 | ND | ND |
| NCI-H446 | High | 10 nM | 168 | ASCL1 | 0.58 | 1.28 |
| NCI-H446 | Low | 0.1 nM | 24 | ASCL1 | −0.27 | 0.74 |
| NCI-H446 | Low | 0.1 nM | 96 | ASCL1 | ND | ND |
| NCI-H446 | Low | 0.1 nM | 168 | ASCL1 | −0.56 | 1.42 |
| NCI-H446 | Vehicle | Vehicle | 24 | GRP | ND | ND |
| NCI-H446 | Vehicle | Vehicle | 96 | GRP | ND | ND |
| NCI-H446 | Vehicle | Vehicle | 168 | GRP | ND | ND |
| NCI-H446 | Medium | 1 nM | 24 | GRP | ND | ND |
| NCI-H446 | Medium | 1 nM | 96 | GRP | ND | ND |
| NCI-H446 | Medium | 1 nM | 168 | GRP | ND | ND |
| NCI-H446 | High | 10 nM | 24 | GRP | ND | ND |
| NCI-H446 | High | 10 nM | 96 | GRP | ND | ND |
| NCI-H446 | High | 10 nM | 168 | GRP | ND | ND |
| NCI-H446 | Low | 0.1 nM | 24 | GRP | ND | ND |
| NCI-H446 | Low | 0.1 nM | 96 | GRP | ND | ND |
| NCI-H446 | Low | 0.1 nM | 168 | GRP | ND | ND |
| NCI-H446 | Vehicle | Vehicle | 24 | NOTCH1 | 0.00 | 0.13 |
| NCI-H446 | Vehicle | Vehicle | 96 | NOTCH1 | −0.11 | 0.09 |
| NCI-H446 | Vehicle | Vehicle | 168 | NOTCH1 | −0.05 | 0.02 |
| NCI-H446 | Medium | 1 nM | 24 | NOTCH1 | −0.02 | 0.15 |
| NCI-H446 | Medium | 1 nM | 96 | NOTCH1 | 0.06 | 0.14 |
| NCI-H446 | Medium | 1 nM | 168 | NOTCH1 | 0.29 | 0.18 |
| NCI-H446 | High | 10 nM | 24 | NOTCH1 | 0.04 | 0.07 |
| NCI-H446 | High | 10 nM | 96 | NOTCH1 | 0.16 | 0.11 |
| NCI-H446 | High | 10 nM | 168 | NOTCH1 | 0.42 | 0.09 |
| NCI-H446 | Low | 0.1 nM | 24 | NOTCH1 | −0.04 | 0.10 |
| NCI-H446 | Low | 0.1 nM | 96 | NOTCH1 | −0.13 | 0.07 |
| NCI-H446 | Low | 0.1 nM | 168 | NOTCH1 | −0.40 | 0.14 |
| NCI-H510A | Vehicle | Vehicle | 24 | ASCL1 | 0.00 | 0.26 |
| NCI-H510A | Vehicle | Vehicle | 96 | ASCL1 | 0.41 | 0.11 |
| NCI-H510A | Vehicle | Vehicle | 168 | ASCL1 | −0.06 | 0.16 |
| NCI-H510A | Medium | 1 nM | 24 | ASCL1 | −0.23 | 0.07 |
| NCI-H510A | Medium | 1 nM | 96 | ASCL1 | −0.50 | 0.12 |
| NCI-H510A | Medium | 1 nM | 168 | ASCL1 | −0.51 | 0.06 |
| NCI-H510A | High | 10 nM | 24 | ASCL1 | −0.28 | 0.13 |
| NCI-H510A | High | 10 nM | 96 | ASCL1 | −0.55 | 0.12 |
| NCI-H510A | High | 10 nM | 168 | ASCL1 | −0.64 | 0.14 |
| NCI-H510A | Low | 0.1 nM | 24 | ASCL1 | 0.07 | 0.04 |
| NCI-H510A | Low | 0.1 nM | 96 | ASCL1 | 0.17 | 0.13 |
| NCI-H510A | Low | 0.1 nM | 168 | ASCL1 | 0.03 | 0.07 |
| NCI-H510A | Vehicle | Vehicle | 24 | GRP | 0.00 | 0.11 |
| NCI-H510A | Vehicle | Vehicle | 96 | GRP | 0.12 | 0.18 |
| NCI-H510A | Vehicle | Vehicle | 168 | GRP | −0.34 | 0.10 |
| NCI-H510A | Medium | 1 nM | 24 | GRP | −0.30 | 0.09 |
| NCI-H510A | Medium | 1 nM | 96 | GRP | −1.75 | 0.03 |
| NCI-H510A | Medium | 1 nM | 168 | GRP | −2.34 | 0.10 |
| NCI-H510A | High | 10 nM | 24 | GRP | −0.35 | 0.03 |
| NCI-H510A | High | 10 nM | 96 | GRP | −2.02 | 0.06 |
| NCI-H510A | High | 10 nM | 168 | GRP | −2.53 | 0.12 |
| NCI-H510A | Low | 0.1 nM | 24 | GRP | 0.22 | 0.03 |
| NCI-H510A | Low | 0.1 nM | 96 | GRP | −0.30 | 0.11 |
| NCI-H510A | Low | 0.1 nM | 168 | GRP | −0.44 | 0.08 |
| NCI-H510A | Vehicle | Vehicle | 24 | NOTCH1 | 0.00 | 0.48 |
| NCI-H510A | Vehicle | Vehicle | 96 | NOTCH1 | 1.48 | 0.13 |
| NCI-H510A | Vehicle | Vehicle | 168 | NOTCH1 | 2.76 | 0.12 |
| NCI-H510A | Medium | 1 nM | 24 | NOTCH1 | 1.19 | 0.56 |
| NCI-H510A | Medium | 1 nM | 96 | NOTCH1 | 2.87 | 0.10 |
| NCI-H510A | Medium | 1 nM | 168 | NOTCH1 | 3.98 | 0.15 |
| NCI-H510A | High | 10 nM | 24 | NOTCH1 | 1.60 | 0.36 |
| NCI-H510A | High | 10 nM | 96 | NOTCH1 | 2.89 | 0.24 |
| NCI-H510A | High | 10 nM | 168 | NOTCH1 | 4.16 | 0.19 |
| NCI-H510A | Low | 0.1 nM | 24 | NOTCH1 | 0.53 | 0.22 |
| NCI-H510A | Low | 0.1 nM | 96 | NOTCH1 | 1.93 | 0.26 |
| NCI-H510A | Low | 0.1 nM | 168 | NOTCH1 | 3.00 | 0.16 |
| NCI-H526 | Vehicle | Vehicle | 24 | ASCL1 | ND | ND |
| NCI-H526 | Vehicle | Vehicle | 96 | ASCL1 | ND | ND |
| NCI-H526 | Vehicle | Vehicle | 168 | ASCL1 | ND | ND |
| NCI-H526 | Medium | 1 nM | 24 | ASCL1 | ND | ND |
| NCI-H526 | Medium | 1 nM | 96 | ASCL1 | ND | ND |
| NCI-H526 | Medium | 1 nM | 168 | ASCL1 | ND | ND |
| NCI-H526 | High | 10 nM | 24 | ASCL1 | ND | ND |
| NCI-H526 | High | 10 nM | 96 | ASCL1 | ND | ND |
| NCI-H526 | High | 10 nM | 168 | ASCL1 | ND | ND |
| NCI-H526 | Low | 0.1 nM | 24 | ASCL1 | ND | ND |
| NCI-H526 | Low | 0.1 nM | 96 | ASCL1 | ND | ND |
| NCI-H526 | Low | 0.1 nM | 168 | ASCL1 | ND | ND |
| NCI-H526 | Vehicle | Vehicle | 24 | GRP | ND | ND |
| NCI-H526 | Vehicle | Vehicle | 96 | GRP | ND | ND |
| NCI-H526 | Vehicle | Vehicle | 168 | GRP | ND | ND |
| NCI-H526 | Medium | 1 nM | 24 | GRP | ND | ND |
| NCI-H526 | Medium | 1 nM | 96 | GRP | ND | ND |
| NCI-H526 | Medium | 1 nM | 168 | GRP | ND | ND |
| NCI-H526 | High | 10 nM | 24 | GRP | ND | ND |
| NCI-H526 | High | 10 nM | 96 | GRP | ND | ND |
| NCI-H526 | High | 10 nM | 168 | GRP | ND | ND |
| NCI-H526 | Low | 0.1 nM | 24 | GRP | ND | ND |
| NCI-H526 | Low | 0.1 nM | 96 | GRP | ND | ND |
| NCI-H526 | Low | 0.1 nM | 168 | GRP | ND | ND |
| NCI-H526 | Vehicle | Vehicle | 24 | NOTCH1 | 0.00 | 0.14 |
| NCI-H526 | Vehicle | Vehicle | 96 | NOTCH1 | 0.54 | 0.05 |
| NCI-H526 | Vehicle | Vehicle | 168 | NOTCH1 | 0.22 | 0.04 |
| NCI-H526 | Medium | 1 nM | 24 | NOTCH1 | 0.27 | 0.05 |
| NCI-H526 | Medium | 1 nM | 96 | NOTCH1 | 0.53 | 0.11 |
| NCI-H526 | Medium | 1 nM | 168 | NOTCH1 | 0.96 | 0.13 |
| NCI-H526 | High | 10 nM | 24 | NOTCH1 | 0.41 | 0.09 |
| NCI-H526 | High | 10 nM | 96 | NOTCH1 | 0.70 | 0.10 |
| NCI-H526 | High | 10 nM | 168 | NOTCH1 | 0.33 | 0.14 |
| NCI-H526 | Low | 0.1 nM | 24 | NOTCH1 | 0.04 | 0.09 |
| NCI-H526 | Low | 0.1 nM | 96 | NOTCH1 | 0.51 | 0.02 |
| NCI-H526 | Low | 0.1 nM | 168 | NOTCH1 | 0.55 | 0.07 |
| NCI-H69 | Vehicle | Vehicle | 24 | ASCL1 | 0.00 | 0.05 |
| NCI-H69 | Vehicle | Vehicle | 96 | ASCL1 | −0.29 | 0.07 |
| NCI-H69 | Vehicle | Vehicle | 168 | ASCL1 | −0.37 | 0.08 |
| NCI-H69 | Medium | 1 nM | 24 | ASCL1 | −0.71 | 0.05 |
| NCI-H69 | Medium | 1 nM | 96 | ASCL1 | −1.33 | 0.08 |
| NCI-H69 | Medium | 1 nM | 168 | ASCL1 | −1.36 | 0.11 |
| NCI-H69 | High | 10 nM | 24 | ASCL1 | −0.80 | 0.01 |
| NCI-H69 | High | 10 nM | 96 | ASCL1 | −1.40 | 0.06 |
| NCI-H69 | High | 10 nM | 168 | ASCL1 | −1.62 | 0.09 |
| NCI-H69 | Low | 0.1 nM | 24 | ASCL1 | −0.47 | 0.04 |
| NCI-H69 | Low | 0.1 nM | 96 | ASCL1 | −0.63 | 0.05 |
| NCI-H69 | Low | 0.1 nM | 168 | ASCL1 | −0.75 | 0.08 |
| NCI-H69 | Vehicle | Vehicle | 24 | GRP | 0.00 | 0.49 |
| NCI-H69 | Vehicle | Vehicle | 96 | GRP | −1.17 | 0.19 |
| NCI-H69 | Vehicle | Vehicle | 168 | GRP | −1.24 | 0.25 |
| NCI-H69 | Medium | 1 nM | 24 | GRP | −0.51 | 0.13 |
| NCI-H69 | Medium | 1 nM | 96 | GRP | −2.14 | 0.16 |
| NCI-H69 | Medium | 1 nM | 168 | GRP | −2.12 | 0.40 |
| NCI-H69 | High | 10 nM | 24 | GRP | −0.97 | 0.18 |
| NCI-H69 | High | 10 nM | 96 | GRP | −2.19 | 0.34 |
| NCI-H69 | High | 10 nM | 168 | GRP | −2.85 | 0.06 |
| NCI-H69 | Low | 0.1 nM | 24 | GRP | −0.77 | 0.24 |
| NCI-H69 | Low | 0.1 nM | 96 | GRP | −1.46 | 0.23 |
| NCI-H69 | Low | 0.1 nM | 168 | GRP | −1.26 | 0.34 |
| NCI-H69 | Vehicle | Vehicle | 24 | NOTCH1 | 0.00 | 0.48 |
| NCI-H69 | Vehicle | Vehicle | 96 | NOTCH1 | 0.17 | 0.82 |
| NCI-H69 | Vehicle | Vehicle | 168 | NOTCH1 | 0.10 | 0.67 |
| NCI-H69 | Medium | 1 nM | 24 | NOTCH1 | 0.26 | 0.46 |
| NCI-H69 | Medium | 1 nM | 96 | NOTCH1 | 2.26 | 0.14 |
| NCI-H69 | Medium | 1 nM | 168 | NOTCH1 | 2.38 | 0.31 |
| NCI-H69 | High | 10 nM | 24 | NOTCH1 | 1.33 | 0.32 |
| NCI-H69 | High | 10 nM | 96 | NOTCH1 | 2.42 | 0.32 |
| NCI-H69 | High | 10 nM | 168 | NOTCH1 | 3.22 | 0.23 |
| NCI-H69 | Low | 0.1 nM | 24 | NOTCH1 | −0.17 | 0.68 |
| NCI-H69 | Low | 0.1 nM | 96 | NOTCH1 | 0.57 | 0.34 |
| NCI-H69 | Low | 0.1 nM | 168 | NOTCH1 | 0.94 | 1.07 |
| SHP-77 | Vehicle | Vehicle | 24 | ASCL1 | 0.00 | 0.11 |
| SHP-77 | Vehicle | Vehicle | 96 | ASCL1 | 0.21 | 0.03 |
| SHP-77 | Vehicle | Vehicle | 168 | ASCL1 | −0.19 | 0.10 |
| SHP-77 | Medium | 1 nM | 24 | ASCL1 | 0.52 | 0.03 |
| SHP-77 | Medium | 1 nM | 96 | ASCL1 | 0.38 | 0.17 |
| SHP-77 | Medium | 1 nM | 168 | ASCL1 | −0.33 | 0.08 |
| SHP-77 | High | 10 nM | 24 | ASCL1 | −0.07 | 0.03 |
| SHP-77 | High | 10 nM | 96 | ASCL1 | 0.22 | 0.14 |
| SHP-77 | High | 10 nM | 168 | ASCL1 | −0.50 | 0.06 |
| SHP-77 | Low | 0.1 nM | 24 | ASCL1 | −0.11 | 0.09 |
| SHP-77 | Low | 0.1 nM | 96 | ASCL1 | 0.11 | 0.07 |
| SHP-77 | Low | 0.1 nM | 168 | ASCL1 | −0.36 | 0.07 |
| SHP-77 | Vehicle | Vehicle | 24 | GRP | 0.00 | 0.09 |
| SHP-77 | Vehicle | Vehicle | 96 | GRP | 1.28 | 0.08 |
| SHP-77 | Vehicle | Vehicle | 168 | GRP | 0.77 | 0.03 |
| SHP-77 | Medium | 1 nM | 24 | GRP | 1.20 | 0.07 |
| SHP-77 | Medium | 1 nM | 96 | GRP | 0.83 | 0.13 |
| SHP-77 | Medium | 1 nM | 168 | GRP | 0.16 | 0.12 |
| SHP-77 | High | 10 nM | 24 | GRP | −0.10 | 0.04 |
| SHP-77 | High | 10 nM | 96 | GRP | 0.56 | 0.13 |
| SHP-77 | High | 10 nM | 168 | GRP | −0.06 | 0.06 |
| SHP-77 | Low | 0.1 nM | 24 | GRP | 0.17 | 0.03 |
| SHP-77 | Low | 0.1 nM | 96 | GRP | 0.74 | 0.17 |
| SHP-77 | Low | 0.1 nM | 168 | GRP | 0.43 | 0.07 |
| SHP-77 | Vehicle | Vehicle | 24 | NOTCH1 | ND | ND |
| SHP-77 | Vehicle | Vehicle | 96 | NOTCH1 | ND | ND |
| SHP-77 | Vehicle | Vehicle | 168 | NOTCH1 | ND | ND |
| SHP-77 | Medium | 1 nM | 24 | NOTCH1 | ND | ND |
| SHP-77 | Medium | 1 nM | 96 | NOTCH1 | ND | ND |
| SHP-77 | Medium | 1 nM | 168 | NOTCH1 | ND | ND |
| SHP-77 | High | 10 nM | 24 | NOTCH1 | ND | ND |
| SHP-77 | High | 10 nM | 96 | NOTCH1 | ND | ND |
| SHP-77 | High | 10 nM | 168 | NOTCH1 | ND | ND |
| SHP-77 | Low | 0.1 nM | 24 | NOTCH1 | ND | ND |
| SHP-77 | Low | 0.1 nM | 96 | NOTCH1 | ND | ND |
| SHP-77 | Low | 0.1 nM | 168 | NOTCH1 | ND | ND |

(ND: not detectable).

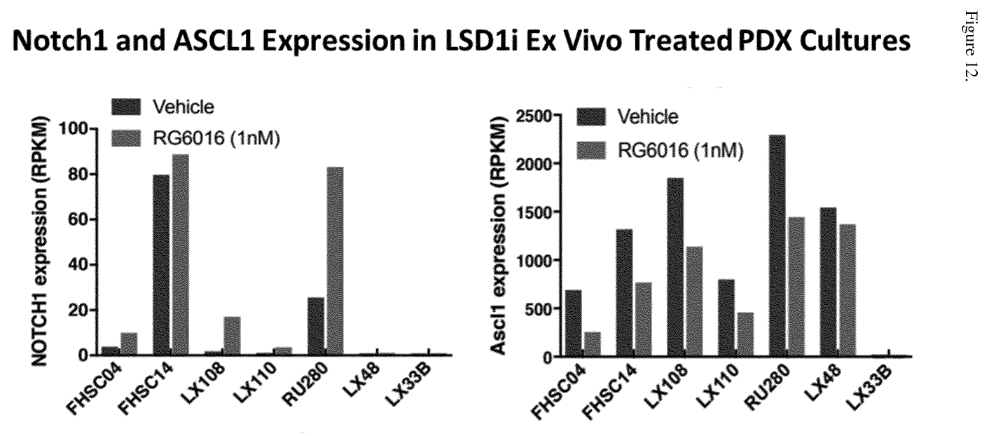

To confirm the results obtained for the panel for small cell lung cancer cell lines, changes in PD markers Notch1 and ASCL1 were evaluated in a panel of patient derived xenograft (PDX) small cell lung cancer models. The initiation and characterization of PDX models LX48, LX108, LX110, LX33 have been described previously (Hann, C. L., et al. 2008, Cancer Research. 68, 2321-2328; Poirier et al. (2013). J. Natl. Cancer Inst. 105, 1059-1065; Leong et al. PLoS One. 2014; 9:e106862). PDX models, FHSC04 and FHSC14 were generated from blood samples obtained from patients with extensive-stage SCLC, following the methodology described by the Dive lab (Hodgkinson et al. 2014 Nat Med August 20(8), 897-903). Ex vivo cultures were treated with 1 nM (trans)-N1-((1R,2S)-2-Phenylcyclopropyl)cyclohexane-1,4-diamine in 96 well plates (10,000 cells/well) for 120 hours. RNA was extracted in TRIzol (Invitrogen) and isolated according to the manufacturer's protocol. cDNA was generated using the iScript synthesis kit (Bio-Rad) according to the manufacturer's protocol. qPCR experiments were run on a Biorad qPCR instrument (CFX384). Data were normalized to GAPDH. FIG. 12 contains the results of the qRT-PCR using the following primers.

NOTCH1-F: GTCAACGCCGTAGATGACC;
NOTCH1-R: TTGTTAGCCCCGTTCTTCAG;
ASCL1-F: GGAGCTTCTCGACTTCACCA;
ASCL1-R: CTAAAGATGCAGGTTGTGCG;
GAPDH-F: CTGGAGAAACCTGCCAAGTA;
GAPDH-R: TGTTGCTGTAGCCGTATTCA

### Example 3. Candidate PD Gene Change in Xenograft Mouse Model

Female athymic nude mice, approximately 7-8-week old animals, were injected with 5 million H510A cells per 50 μL PBS and 50 μL Matrigel (BD Bioscience) into the right flank of each animal. After tumor growth reached 200-300 mm3, animals were distributed in three homogeneous groups with similar mean tumor volume and SD. Animals were treated with vehicle, 20 μg/Kg and 40 μg/Kg of (trans)-N1-((1R,2S)-2-phenylcyclopropyl)cyclohexane-1,4-diamine for 3 weeks according to the 5 days on/2 days off schedule.

Mice were sacrificed at day 35 at 1, 6 and 24 hours post last injection. Blood was extracted in Microvette® (Sarstedt) tubes, centrifuged on a bench-top centrifuge at 2000 rpm for 15 min at 4 C. Supernatant was stored at −80° C. Tumors were removed and divided in 3 parts: ⅓ was immersed in RNA-Later and snap frozen for subsequent analysis. Frozen material was stored at −80° C. before shipment.

Whole transcriptomic RNA sequencing procedure is identical to that as described in Example 1. Raw RNASeq reads were aligned to the mouse and the human transcriptome using GSNAP and were then analyzed. Reads mapped to both organisms were filtered out and only reads that were uniquely aligned to human or mouse were used to profile the respective genes. The log 2 fold change of three candidate PD markers between (trans)-N1-((1R,2S)-2-phenylcyclopropyl)cyclohexane-1,4-diamine treated and vehicle treated samples were calculated using DESeq2 package (Love et al.).

Table 7 shows the dosage- and time-dependent expression change for candidate PD markers ASCL1, GRP and NOTCH1.

TABLE 7
Dosage- and time-dependent expression log2 fold change for candidate
PD markers in SCLC xenograft mouse model (LSD1i indicates
treatment with (trans)-N1-((1R,2S)-2-phenylcyclopropyl)cyclohexane-
1,4-diamine).

| | | | NOTCH1 |
|---|---|---|---|
| | ASCL1 Log2FC | GRP Log2FC | Log2FC |
| Log Ratio 1 h:Vehicle | −0.525 | −1.607 | 1.947 |
| vs LSD1i | | | |
| 20 μg/kg | | | |
| Log Ratio 1 h:Vehicle | −0.783 | −1.75 | 2.727 |
| vs LSD1i | | | |
| 40 μg/kg | | | |
| Log Ratio 6 h:Vehicle | −0.545 | −0.794 | 1.388 |
| vs LSD1i | | | |
| 20 μg/kg | | | |
| Log Ratio 6 h:Vehicle | −0.825 | −1.421 | 2.321 |
| vs LSD1i | | | |
| 40 μg/kg | | | |
| Log Ratio 24 h:Vehicle | −0.472 | −1.205 | 1.679 |
| vs LSD1i | | | |
| 20 μg/kg | | | |
| Log Ratio 24 h:Vehicle | −1.286 | −2.742 | 3.423 |
| vs LSD1i | | | |
| 40 μg/kg | | | |

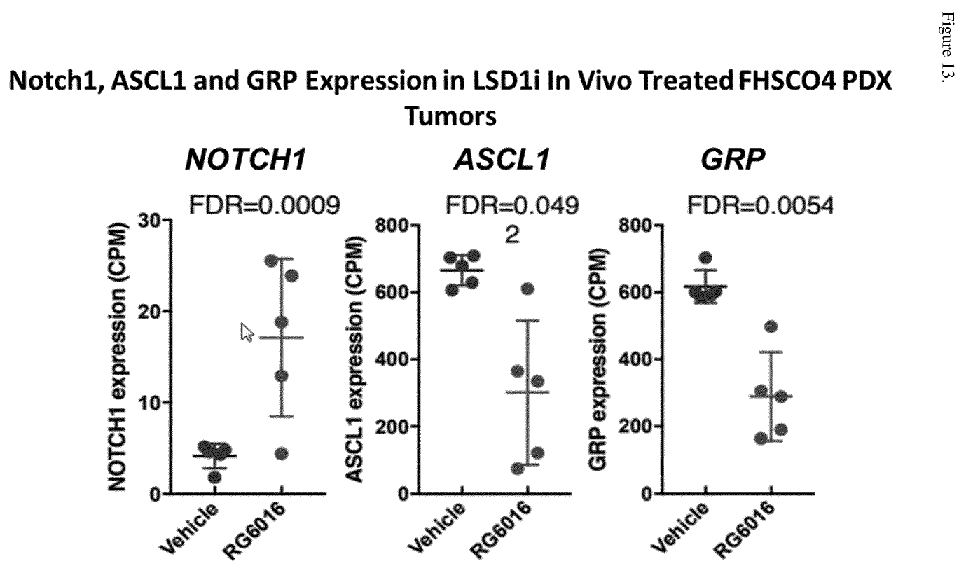

To confirm the results obtained for small cell lung cancer cell line xenograft, changes in PD markers Notch1, ASCL1 and GRP were evaluated in PDX model FHSC04 treated in vivo with LSD1i (trans)-N1-((1R,2S)-2-phenylcyclopropyl)cyclohexane-1,4-diamine (FIG. 13). Eight to twelve-week-old NOD scid gamma mice were implanted with 1.0×10⁶ disaggregated cells from PDX model FHSC04 in 100 μl of 1:1 HITES media:Matrigel. Once palpable, flank tumors were measured in two dimensions (length and width) using digital calipers and volume was calculated using the formula for a prolate ellipsoid, length (mm)×width (mm)/2=mm³. Once tumor volume reached 150-200 mm³, mice were randomly assigned to treatment groups for 21-35 days, depending on the kinetics of growth in the given model. Animals were treated with saline or with 400 μg/kg ((trans)-N1-((1R,2S)-2-phenylcyclopropyl)cyclohexane-1,4-diamine, delivered by oral gavage once every 7 days, dissolved to 10 ml/kg with 0.9% saline solution. For the 10-day treatment in FHSC04 used for molecular analyses, an additional dose of 400 μg/kg (trans)-N1-((1R,2S)-2-phenylcyclopropyl)cyclohexane-1,4-diamine was given 4 hours before tissue collection.

For RNA-seq analyses using PDX samples in vivo, the Ultra RNA Library Prep Kit for Illumina (New England BioLabs; catalog E753L) was used to generate libraries from total RNA. All library preparation was conducted according to the manufacturer's instructions. Single-end sequencing (50 bp) was performed using an Illumina HiSeq 2500, reads of low quality were filtered prior to alignment to the hg19 genome build using TopHat v2.0.12 (Trapnell et al. Bioinformatics 2009 May 1; 25(9): 1105-1111). In vivo PDX samples were also aligned to mm9, where the mouse and human alignments from a given sample were compared, discarding from downstream analysis those reads which had fewer mismatches to mouse, relative to human. Cuffdiff v2.1.1 (Trapnell et al. Nat Biotechnol. 2013 January; 31(1):46-53) was used to generate fragment per kilobase per million (FPKM) expression values. Counts (CPM) were generated from TopHat alignments using the Python package HTSeq v0.6.1 (Anders et al. Bioinformatics. 2015 Jan. 15; 31(2): 166-169) using the “intersection-strict” overlap mode. Genes with low counts across conditions were discarded prior to identification of differentially expressed genes using the Bioconductor package edgeR, v3.16.5 (Robinson et al. 2010 Jan. 1; 26(1):139-40). A false discovery rate (FDR) (Reiner et al. (2003) Bioinformatics, 19(3), 368-375.) method was used to correct for multiple testing, where differentially expressed genes were identified with the FDR set at 5%. GOTERM_BP_DIRECT ontologies were acquired from DAVID v6.8 (Huang da et al. Nat Protoc, 2009; 4(1):44-57), and used with the Bioconductor package goseq v1.26.0 (Young et al. 2010, Genome Biology 11:R14) to identify overrepresented genes that were either significantly up or downregulated, applying a FDR method to correct for multiple testing (Reiner et al. 2003).

1. An in vitro method of assessing the response of a patient having a neoplastic disease to a therapy comprising an LSD1 inhibitor, the method comprising steps:
a) prior to begin of the therapy measuring in a sample from the patient one or more mRNA transcript expression levels of a gene panel and/or one or more expression levels of the translated proteins of a gene panel, wherein the gene panel comprises one or more genes,
b) after begin of the therapy measuring in a sample from the patient the levels as measured in a) of the gene panel,
c) comparing the levels of the gene panel measured in a) to the levels of the gene panel measured in b), and
d) identifying the patient as responding to the therapy when the levels of the gene panel measured in b) are up-regulated or down-regulated as compared to the levels of the gene panel measured in a).

2. The method of claim 1 further comprising step:
e) optimizing the therapy by recommending that the patient be treated with an adapted effective amount of LSD1 inhibitor.

3. An in vitro method of monitoring efficacy of therapy comprising an LSD1 inhibitor in a patient having a neoplastic disease, the method comprising steps a), b), c) and d) according to claim 1.

4. The method of claim 3 further comprising step e) according to claim 2.

5. A method of treating a patient having a neoplastic disease, the method comprising steps a), b), c) and d) according to claim 1 and optionally step e) according to claim 2, and further step:
f) administering the adapted effective amount of LSD1 inhibitor to the patient if likely to respond thereby treating the neoplastic disease.

6. An LSD1 inhibitor for use in treating a patient having a neoplastic disease, wherein the patient is treated if one or more mRNA transcript expression levels of a gene panel and/or one or more expression levels of the translated proteins of a gene panel measured in a sample from the patient after begin of the therapy are up-regulated or down-regulated as compared to the levels measured prior to begin of the therapy thereby treating the neoplastic disease, wherein the gene panel comprises one or more genes.

7. An in vitro use of a gene panel comprising one or more genes for assessing a therapy comprising an LSD1 inhibitor in a patient having a neoplastic disease, wherein up-regulation or down-regulation of one or more mRNA transcript expression levels of a gene panel and/or one or more expression levels of the translated proteins of a gene panel measured in a sample from the patient after begin of the therapy as compared to the levels measured prior to begin of the therapy indicate that the patient should be treated with an effective amount of an LSD1 inhibitor.

8. An in vitro use of a gene panel comprising one or more genes for monitoring efficacy of therapy comprising an LSD1 inhibitor in a patient having a neoplastic disease, wherein up-regulation or down-regulation of one or more mRNA transcript expression levels of a gene panel and/or one or more expression levels of the translated proteins of a gene panel measured in a sample from the patient after begin of the therapy as compared to the levels measured prior to begin of the therapy indicate that the patient should be treated with an effective amount of an LSD1 inhibitor.

9. Use of a gene panel comprising one or more genes for the manufacture of a diagnostic for assessing a neoplastic disease.

10. Use of a gene panel comprising one or more genes for the manufacture of a diagnostic for assessing a therapy comprising an LSD1 inhibitor in a patient having a neoplastic disease.

11. Use of a gene panel comprising one or more genes for the manufacture of a diagnostic for monitoring efficacy of therapy comprising an LSD1 inhibitor in a patient having a neoplastic disease.

12. A kit for monitoring efficacy of therapy comprising an LSD1 inhibitor in a patient having a neoplastic disease comprising one or more reagents for measuring one or more mRNA transcript expression levels of a gene panel and/or one or more expression levels of the translated proteins of a gene panel in a sample, wherein the gene panel comprises one or more genes.

13. The method according to any of claims 1 to 5, the LSD1 inhibitor of claim 6, the use according to any of claims 7 to 11, or the kit of claim 12, wherein the levels measured are mRNA transcript expression levels.

14. The method according to any of claims 1 to 5, the LSD1 inhibitor of claim 6, the use according to any of claims 7 to 11, or the kit of claim 12, wherein the levels measured are mRNA transcript expression levels derived from RNA-sequencing, RT-qPCR or microarrays.

15. The method according to any of claims 1 to 5, the LSD1 inhibitor of claim 6, the use according to any of claims 7 to 11, or the kit of claim 12, wherein the levels measured are expression levels of translated proteins.

16. The method according to any of claims 1 to 5, the LSD1 inhibitor of claim 6, the use according to any of claims 7 to 11, or the kit of claim 12, wherein the gene panel comprises one or more genes selected from the group of NOTCH1, ASCL1, GRP, CNN2, DENND5A, VIM, and ZFP36L1.

17. The method according to any of claims 13 to 15, the LSD1 inhibitor of claim 6, the use according to any of claims 7 to 11, or the kit of any one of claims 12 to 15, wherein the gene panel comprises one or more genes selected from the group of NOTCH1, ASCL1, GRP, CNN2, DENND5A, VIM, and ZFP36L1.

18. The method according to any of claims 1 to 5, the LSD1 inhibitor of claim 6, the use according to any of claims 7 to 11, or the kit of claim 12, wherein the gene panel comprises one or more genes selected from the group of NOTCH1, CNN2, DENND5A, VIM, and ZFP36L1.

19. The method according to any of claims 13 to 15, the LSD1 inhibitor of claim 6, the use according to any of claims 7 to 11, or the kit of claim 12, wherein the gene panel comprises one or more genes selected from the group of NOTCH1, CNN2, DENND5A, VIM, and ZFP36L1.

20. The method according to any of claims 1 to 5, the LSD1 inhibitor of claim 6, the use according to any of claims 7 to 11, or the kit of claim 12, wherein the gene panel comprises one or more genes selected from the group of NOTCH1, ASCL1, GRP, CNN2, DENND5A, and ZFP36L1.

21. The method according to any of claims 13 to 15, the LSD1 inhibitor of claim 6, the use according to any of claims 7 to 11, or the kit of claim 12, wherein the gene panel comprises one or more genes selected from the group of NOTCH1, ASCL1, GRP, CNN2, DENND5A, and ZFP36L1.

22. The method according to any of claims 1 to 5, the LSD1 inhibitor of claim 6, the use according to any of claims 7 to 11, or the kit of claim 12, wherein the gene panel comprises one or more genes selected from the group of NOTCH1, CNN2, DENND5A, and ZFP36L1.

23. The method according to any of claims 13 to 15, the LSD1 inhibitor of claim 6, the use according to any of claims 7 to 11, or the kit of claim 12, wherein the gene panel comprises one or more genes selected from the group of NOTCH1, CNN2, DENND5A, and ZFP36L1.

24. The method according to any of claims 1 to 5, the LSD1 inhibitor of claim 6, the use according to any of claims 7 to 11, or the kit of claim 12, wherein the gene panel comprises or more genes selected from the group of NOTCH1, ASCL1, GRP, CNN2, DENND5A, VIM, and ZFP36L1, wherein up-regulated levels of NOTCH1, CNN2, DENND5A, VIM, and ZFP36L1 and/or down-regulated levels of ASCL1 and GRP after begin of therapy comprising an LSD1 inhibitor are indicative for a response of the patient to the therapy.

25. The method according to any of claims 13 to 15, the LSD1 inhibitor of claim 6, the use according to any of claims 7 to 11, or the kit of claim 12, wherein the gene panel comprises or more genes selected from the group of NOTCH1, ASCL1, GRP, CNN2, DENND5A, VIM, and ZFP36L1, wherein up-regulated levels of NOTCH1, CNN2, DENND5A, VIM, and ZFP36L1 and/or down-regulated levels of ASCL1 and GRP after begin of therapy comprising an LSD1 inhibitor are indicative for a response of the patient to the therapy.

26. The method according to any of claims 1 to 5 or 13 to 15, the LSD1 inhibitor of claim 6, in particular (trans)-N1-((1R,2S)-2-phenylcyclopropyl)cyclohexane-1,4-diamine, the use according to any of claims 7 to 11, or the kit of claim 12, wherein the gene panel comprises the NOTCH1 gene, wherein up-regulated levels of NOTCH1 after begin of therapy comprising the LSD1 inhibitor of claim 6, in particular (trans)-N1-((1R,2S)-2-phenylcyclopropyl)cyclohexane-1,4-diamine, are indicative for a response of the patient to the therapy.

27. The method according to any of claims 1 to 5, the LSD1 inhibitor of claim 6, the use according to any of claims 7 to 11, or the kit of claim 12, wherein the gene panel consists of one, two, three, four or five genes.

28. The method according to any of claims 13 to 15, the LSD1 inhibitor of claim 6, the use according to any of claims 7 to 11, or the kit of claim 12, wherein the gene panel consists of one, two, three, four or five genes.

29. The method according to any of claims 1 to 5, the LSD1 inhibitor of claim 6, the use according to any of claims 7 to 11, or the kit of claim 12, wherein the gene panel consists of two, three or four genes.

30. The method according to any of claims 13 to 15, the LSD1 inhibitor of claim 6, the use according to any of claims 7 to 11, or the kit of claim 12, wherein the gene panel consists of two, three or four genes.

31. The method according to any of claims 1 to 5, the LSD1 inhibitor of claim 6, the use according to any of claims 7 to 11, or the kit of claim 12, wherein the LSD1 inhibitor is selected from the list of:
4-[[4-[[[(1R,2S)-2-phenylcyclopropyl]amino]methyl]-1-piperidinyl]methyl]-benzoic acid, (trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-diamine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine,
and a pharmaceutically acceptable salt thereof.

32. The method according to any of claims 13 to 30, the LSD1 inhibitor of claim 6, the use according to any of claims 7 to 11, or the kit of claim 12, wherein the LSD1 inhibitor is selected from the list of:
4-[[4-[[[(1R,2S)-2-phenylcyclopropyl]amino]methyl]-1-piperidinyl]methyl]-benzoic acid,
(trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-diamine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine,
and a pharmaceutically acceptable salt thereof.

33. The method according to any of claims 1 to 5, the LSD1 inhibitor of claim 6, the use according to any of claims 7 to 11, or the kit of claim 12, wherein the LSD1 inhibitor is (trans)-N1-((1R,2S)-2-phenylcyclopropyl)cyclohexane-1,4-diamine or a pharmaceutically acceptable salt thereof.

34. The method according to any of claims 13 to 30, the LSD1 inhibitor of claim 6, the use according to any of claims 7 to 11, or the kit of claim 12, wherein the LSD1 inhibitor is (trans)-N1-((1R,2S)-2-phenylcyclopropyl)cyclohexane-1,4-diamine or a pharmaceutically acceptable salt thereof.

35. The method according to any of claims 1 to 5, the LSD1 inhibitor of claim 6, the use according to any of claims 7 to 11, or the kit of claim 12, wherein the LSD1 inhibitor is (trans)-N1-((1R,2S)-2-phenylcyclopropyl)cyclohexane-1,4-diamine bis-hydrochloride.

36. The method according to any of claims 13 to 30, the LSD1 inhibitor of claim 6, the use according to any of claims 7 to 11, or the kit of claim 12, wherein the LSD1 inhibitor is (trans)-N1-((1R,2S)-2-phenylcyclopropyl)cyclohexane-1,4-diamine bis-hydrochloride.

37. The method according to any of claims 1 to 5, the LSD1 inhibitor of claim 6, the use according to any of claims 7 to 8, or the kit of claim 12, wherein the sample is taken from a whole blood specimen, a blood serum specimen, a blood plasma specimen, a bone marrow specimen, or a fresh, frozen or formalin-fixed paraffin embedded primary human tumor specimen.

38. The method according to any of claims 13 to 30, the LSD1 inhibitor of claim 6, the use according to any of claims 7 to 8, or the kit of claim 12, wherein the sample is taken from a whole blood specimen, a blood serum specimen, a blood plasma specimen, a bone marrow specimen, or a fresh, frozen or formalin-fixed paraffin embedded primary human tumor specimen.

39. The method according to any of claims 1 to 5 or 13 to 30, the LSD1 inhibitor of claim 6, in particular (trans)-N1-((1R,2S)-2-phenylcyclopropyl)cyclohexane-1,4-diamine, the use according to any of claims 7 to 8, or the kit of claim 12, wherein the sample is taken from a whole blood specimen, a blood serum specimen, a blood plasma specimen, a bone marrow specimen, a saliva specimen, a skin specimen, a hair specimen, a fresh, frozen or formalin-fixed paraffin embedded primary human tumor specimen, a fresh, frozen or formalin-fixed paraffin embedded non-primary tumors, in particular metastases, ascites or circulating tumor cells.

40. The method according to any of claims 1 to 5, the LSD1 inhibitor of claim 6, the use according to any of claims 7 to 11, or the kit of claim 12, wherein the neoplastic disease is a cancer selected from the group consisting of breast cancer, prostate cancer, cervical cancer, ovarian cancer, gastric cancer, colorectal cancer, pancreatic cancer, liver cancer, brain cancer, neuroendocrine cancer, lung cancer, kidney cancer, hematological malignancies, melanoma and sarcoma.

41. The method according to any of claims 13 to 30, the LSD1 inhibitor of claim 6, the use according to any of claims 7 to 11, or the kit of claim 12, wherein the neoplastic disease is a cancer selected from the group consisting of breast cancer, prostate cancer, cervical cancer, ovarian cancer, gastric cancer, colorectal cancer, pancreatic cancer, liver cancer, brain cancer, neuroendocrine cancer, lung cancer, kidney cancer, hematological malignancies, melanoma and sarcoma.

42. The method according to any of claims 1 to 5, the LSD1 inhibitor of claim 6, the use according to any of claims 7 to 11, or the kit of claim 12, the neoplastic disease is a blood cancer or lung cancer selected from the group of acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

43. The method according to any of claims 13 to 30, the LSD1 inhibitor of claim 6, the use according to any of claims 7 to 11, or the kit of claim 12, the neoplastic disease is a blood cancer or lung cancer selected from the group of acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

44. The method according to any of claims 1 to 5, the LSD1 inhibitor of claim 6, the use according to any of claims 7 to 11, or the kit of claim 12, wherein the neoplastic disease is a cancer selected from the group consisting of acute myeloid leukemia (AML), thyroid cancer, melanoma, or small cell lung cancer (SCLC).

45. The method according to any of claims 13 to 30, the LSD1 inhibitor of claim 6, the use according to any of claims 7 to 11, or the kit of claim 12, wherein the neoplastic disease is a cancer selected from the group consisting of acute myeloid leukemia (AML), thyroid cancer, melanoma, or small cell lung cancer (SCLC).

46. The method according to any of claims 1 to 5, the LSD1 inhibitor of claim 6, the use according to any of claims 7 to 11, or the kit of claim 12, wherein the neoplastic disease is small cell lung cancer (SCLC).

47. The method according to any of claims 13 to 30, the LSD1 inhibitor of claim 6, the use according to any of claims 7 to 11, or the kit of claim 12, wherein the neoplastic disease is small cell lung cancer (SCLC).

48. The method according to any of claims 1 to 5, the LSD1 inhibitor of claim 6, the use according to any of claims 7 to 11, or the kit of claim 12, wherein the LSD1 inhibitor is 4-[[4-[[[(1R,2S)-2-phenylcyclopropyl]amino]methyl]-1-piperidinyl]methyl]-benzoic acid or a pharmaceutically acceptable salt thereof.

49. The method according to any of claims 13 to 30, the LSD1 inhibitor of claim 6, the use according to any of claims 7 to 11, or the kit of claim 12, wherein the LSD1 inhibitor is 4-[[4-[[[(1R,2S)-2-phenylcyclopropyl]amino]methyl]-1-piperidinyl]methyl]-benzoic acid or a pharmaceutically acceptable salt thereof.

50. The invention as hereinbefore described.


SEQUENCE LISTING (6 sequences)

SEQ ID NO: 1 - DNA, 19 bp, Artificial Sequence
  modified peptide
gtcaacgccgtagatgacc

SEQ ID NO: 2 - DNA, 20 bp, Artificial Sequence
  modified peptide
ttgttagccccgttcttcag

SEQ ID NO: 3 - DNA, 20 bp, Artificial Sequence
  modified peptide
ggagcttctcgacttcacca

SEQ ID NO: 4 - DNA, 20 bp, Artificial Sequence
  modified peptide
ctaaagatgcaggttgtgcg

SEQ ID NO: 5 - DNA, 20 bp, Artificial Sequence
  modified peptide
ctggagaaacctgccaagta

SEQ ID NO: 6 - DNA, 20 bp, Artificial Sequence
  modified peptide
tgttgctgtagccgtattca